(19) United States
(12) Patent Application Publication
(10) Pub. No.: US 2014/0330100 A1
(43) Pub. Date: Nov. 6, 2014

(54) CARBON NANOSTRUCTURE ELECTROCHEMICAL SENSOR AND METHOD

(71) Applicants: Chunhong Li, Chester Springs, PA (US); David J. Ruggieri, Flourtown, PA (US)

(72) Inventors: Chunhong Li, Chester Springs, PA (US); David J. Ruggieri, Flourtown, PA (US)

(73) Assignee: NANOSELECT, INC., Wilmington, DE (US)

(21) Appl. No.: 14/344,018

(22) PCT Filed: Sep. 10, 2012
(86) PCT No.: PCT/US2012/054399

§ 371 Date: Jul. 1, 2014

Related U.S. Application Data
(60) Provisional application No. 61/533,310, filed on Sep. 12, 2011.

(51) Int. Cl. G01N 27/26; A61B 5/145; A61B 5/1486; G01N 27/30; G01N 27/327

(52) U.S. Cl. CPC G01N 27/26; G01N 27/302; G01N 27/327; A61B 5/1486; A61B 5/14539; Y10S 977/742; Y10S 977/752; Y10S 977/75; Y10S 977/902; B82Y 40/00
USPC 600/345; 204/433; 204/403.01; 204/403.14; 600/309; 427/113; 977/742; 977/752; 977/750; 977/904; 977/847

(57) ABSTRACT

Carbon nanostructures may be protected and functionalized using a layer-by-layer method whereby functional groups on the carbon nanostructure surface may be further derivatized to incorporate additional functional moieties. Exemplary moieties include redox mediator molecules, crown ethers, catalysts, boric acids, carbohydrates, oligonucleotides, DNA or RNA aptamers, peptide aptamers, proteins such as enzymes and antibodies, quantum dots and nanoparticles, cells, cell organelles, or other cellular components. The density of functional groups or functional moieties on carbon nanostructure surfaces may also be controlled as well as the degree of surface hydrophilicity of the nanostructure. Carbon nanostructures functionalized using such a layer-by-layer method may be used to disperse, sort, separate and purify carbon nanostructures and may be used as sensing elements such as voltammetric, amperometric, and potentiometric pH sensors or as biometric sensing elements and electrodes and intracorporeal sensors and electrode.

### CROSS-REFERENCE TO RELATED APPLICATIONS

The present application is co-pending with and claims the priority benefit of the provisional application entitled “Layer-by-Layer Carbon Nanostructure Surface Functionalization and the Applications of Such Functionalized Nanostructures,” Application Ser. No. 61/533,310, filed on Sep. 12, 2011, the entirety of which is incorporated herein by reference. The present application is co-pending with and related to the non-provisional application entitled, “Carbon Nanostructure Electrochemical Sensor and Method,” application Ser. No. ______, filed on Sep. ______, 2012.

### FIELD OF DISCLOSURE

The disclosed system and method generally relate to a layer-by-layer surface functionalization of carbon nanostructures. More specifically, the disclosed system and method relate to layer-by-layer surface functionalization of carbon nanostructures and the application of such functionalized carbon nanostructures as sensing elements.

### BACKGROUND

Interest has grown in utilizing carbon nanostructures including carbon nanotubes (CNT) in sensing applications due to the unique mechanical, electrical and optical properties of CNTs. Pristine CNTs are generally hydrophobic and individual CNTs tend to bundle together due to van der Waals forces. Efforts have been made to covalently graft chemical functions to the surface of CNTs for various applications in attempts to impart new properties to these CNTs. For example, one conventional practice to impart new properties to CNTs is to first oxidize CNT powder in HNO₃ solution or oxygen plasma resulting in —OH and —COOH groups on the CNT surface. Functional groups may then be introduced via amide or ester bond formation. These as-functionalized CNTs may then be used as a powder or in a composite to enhance electrical or mechanical properties in various applications. While a viable approach to introduce —OH and —COOH groups on a CNT surface, an oxidation of the CNT surface may damage the CNT tube structure. Further, some C═C double bonds in the CNT may be altered or broken for any type of subsequent covalent functionalization on the CNT surface thereby leaving pits in the CNT surface structure and modifying both the mechanical and electrical properties of the CNT. Furthermore, such a conventional process does not readily control the degree of functionalization and the density of the introduced functional groups.

Another conventional practice to impart new properties to CNTs includes non-covalent functionalization of CNTs such as dispersion and solubilization of CNT powders using surfactants and polymers. WO 03/050332 describes a preparation of CNT dispersions in liquid, WO 02/16257 describes a polymer wrapped, single-walled CNT, WO 03/102020 describes a method for obtaining peptides that bind to a CNT and other carbon nanostructures, WO 02/095099 describes non-covalent sidewall functionalization of CNTs, and WO 07/013,872 describes the use of non-covalently functionalized CNTs as a sensing composition. Generally, the non-covalent approach relies upon favorable interactions between adsorbed molecules and CNT sidewalls, namely, van der Waals, π-π, and CH-π interactions. As no covalent bond is formed between the adsorbed molecules and CNT sidewalls, non-covalent functionalization of CNTs using these conventional approaches most likely results in little disturbance to the it system in a CNT and thus minimal alteration to the mechanical, electrical and spectroscopic properties of CNTs. These non-covalent approaches typically perform well in dispersing and solubilizing CNT powders, and such approaches generally include mechanical force processes such as ultrasonication and/or mechanical milling to form such powders.

An additional conventional approach to impart new properties to harness the superior properties of CNTs is to grow CNTs on a substrate, functionalize these CNTs on the substrate, and then use the resulting carbon nanostructure on the substrate as an electrode material. For example, a thin film of a metal catalyst such as nickel, cobalt or iron may first be deposited on a silicon substrate with a titanium adhesion or barrier layer. This film may then be annealed at high temperature leading to the formation of small metal particles on the substrate. Feed gases such as acetylene, hydrogen and argon are introduced and contact the surface of each particle of metal catalyst whereby CNTs grow from the particles. The metal catalyst particles may then serve as conducting contacts between the CNTs and the substrate. These grown CNTs, however, are unstable toward strongly oxidizing agents including HNO₃. For example, when these grown CNTs are treated with an HNO₃ solution, CNTs are lost from the substrate as the metal catalyst is oxidized and consumed. Therefore, to maintain the structural integrity of the carbon nanostructure on a substrate and the electric contact between a CNT and the substrate, the metal catalyst particles must first be protected from strong oxidizing agents and/or the functionalization of CNTs on a respective substrate conducted under mild conditions.

While conventional non-covalent processes described above may result in the protection and functionalization of carbon nanostructures, it is unclear what surfactants, polymers or peptides are suitable for various applications. Further, if mechanical force processes such as ultrasonication are required for the non-covalent functionalization, such processes are unsuitable for carbon nanostructures on a substrate as these structures are known to peel off from the substrate upon ultrasonication. Furthermore, when CNTs are dispersed and functionalized with general surfactants and polymers, the control of the deposition thickness of surfactants and/or polymers is difficult. Additionally, most surfactants and polymers used for CNT dispersion and functionalization are non-conducting and introduce an uncontrolled amount of foreign materials (e.g., conducting or non-conducting) to the respective CNT surface which may compromise any superior electrical properties of the CNT. Thus, for sensing applications, it is important that functionalized carbon nanostructures are free from non-specific adsorption. For example, serum albumin, an abundant plasma protein in mammal, forms complexes with CNT whereby the binding leads to quenching of the band gap fluorescence of CNT. An uncontrolled thickness of surface deposition of polymers or proteins may effectively block access to or shield the CNT from the environment. Thus, in such instances, the CNT would cease to function as sensing element. Therefore, a need exists in the art for a well-controlled surface functionalization of carbon nanostructures without altering the superior properties of such carbon nanostructures and provide exemplary nanostructures for biometric and other industry usage. Thus, it is desirable to overcome the limitations of the prior art and provide a carbon nanostructure having functionalized layers and utilize such structures as sensors and the like.

### SUMMARY

Embodiments of the present subject matter may protect and functionalize carbon nanostructures using a layer-by-layer approach. For example, various functional groups and functional moieties may be introduced onto the carbon nanostructure surface platform thereby resulting in carbon nanostructures suitable for various applications. Embodiments of the present subject matter may also control the thickness of the functionalization layer thereby resulting in minimal alteration of the intrinsic electrical and optical properties of such carbon nanostructures. Additionally, embodiments of the present subject matter may adjust the density of introduced functional groups and functional moieties and may modulate the degree of surface hydrophilicity of the functionalized carbon nanostructures. Functionalized carbon nanostructures formed according to exemplary embodiments may then be stable and robust in resisting fouling (e.g., mineral deposition and biofouling) when used in aqueous applications. For example, one embodiment of the present subject matter includes a stable CNT electrochemical sensor which is adaptable to determine free chlorine, bromine, chlorine dioxide and ozone concentrations in flowing tap water.

Another embodiment of the present subject matter finds applicability as a voltammetric pH sensor when a pH responsive redox mediator moiety is introduced onto a CNT surface. This resulting CNT electrode may then be used in a buffer solution with high ionic strength and/or a non-buffered tap water solution. A further embodiment of the present subject matter provides a functionalized CNT-based potentiometric pH sensor for flowing tap water with low conductivity. Such an exemplary functionalized CNT electrode may be employed to monitor molecular binding or interaction events on an electrode surface in electrochemical impedance spectroscopy or in a field-effect transistor (FET) device (e.g., ion-selective FET and solution-gate FET). Pristine CNTs may also be dispersed and functionalized using an exemplary layer-by-layer approach for CNT sorting, separation and purification; and, exemplary surface functionalized CNTs according to embodiments of the present subject matter may be utilized as optical sensors by harnessing the unique spectroscopic properties of CNT such as optical absorption, luminescence and Raman scattering.

One embodiment of the present subject matter provides a layer-by-layer protection and functionalization of a carbon nanostructure by subjecting carbon nanostructures to a composition comprising an alkyl protective moiety under conditions that permit the formation of an alkyl protective moiety layer adjacent to the carbon nanostructure. Various functional groups and functional moieties may subsequently be introduced to form a second layer above the alkyl protective moiety layer. These introduced functional groups and functional moieties may, in other embodiments, undergo further transformations to incorporate additional layers and/or functionalities to the respective carbon nanostructure surface.

A further embodiment of the present subject matter provides a method for the protection of carbon nanostructures. The method may include protecting the surface of such a structure, e.g., a carbon and metal catalyst on a substrate, by contacting the nanostructures with a composition comprising an alkyl protective moiety under conditions that permit the formation of an alkyl protective moiety layer disposed directly adjacent to at least a portion of the metal catalyst, the carbon, or both.

An additional embodiment of the present subject matter provides a method for subsequent surface functionalization of carbon nanostructures comprising forming a second layer and/or third layer on nanostructures that have been protected with an alkyl protective moiety layer. Exemplary functionalization may include, but is not limited to, the introduction of various functional groups such as —OH, —COOH, —NH₂, —NHR, —SH, —S—S—R, —C≡CH, —N₃, —CN, —CHO, —CONH—NH₂, a maleimido group, epoxide, and other functional moieties such as redox mediator structures. Of course, these functional groups may be further derivatized to form covalent bonds with other functional moieties including, but not limited to, redox mediator molecules, crown ethers, catalysts, boric acids, carbohydrates, oligonucleotides (DNAs and RNAs), DNA or RNA aptamers, peptide aptamers, proteins such as enzymes and antibodies, quantum dots and nanoparticles, cells, cell organelles, and other cellular components, either before or after the formation of the second layer on carbon nanostructure surfaces. Thus, such a disclosure should not limit the scope of the claims appended herewith.

Embodiments of the present subject matter may also control the density of specific functional groups and functional moieties on carbon nanostructure surfaces and may control the degree of hydrophilicity of functionalized carbon nanostructure surfaces. Exemplary methods may be provided to construct a hydrophilic platform on the surface of a CNT and carbon nanostructure. Functional groups and/or moieties such as redox mediator molecules, crown ethers, catalysts, boric acids, carbohydrates, oligonucleotides (DNAs and RNAs), DNA or RNA aptamers, peptide aptamers, proteins such as enzymes and antibodies, quantum dots and nanoparticles, cells, cell organelles, and other cellular components, may then be introduced on the hydrophilic surface via covalent bond formation that is free from non-specific adsorption.

Exemplary devices formed using embodiments of the present subject matter may include, but are not limited to, voltammetric pH sensors using a layer-by-layer functionalization of carbon nanostructure surface having, for example, redox mediator molecules that require proton participation for their redox reactions (redox peak potential shift and the solution pH adhere to the Nernst Equation), a surface-functionalized CNT-based potentiometric pH sensor, and an amperometric pH sensor, to name a few.

Another embodiment of the present subject matter provides a method of functionalizing carbon nanostructures. The method may include providing a carbon nanostructure having a first protective layer on a surface of the structure and forming a functional second layer over the first protective layer, where the second layer comprises a bipolar molecule with functional groups or functional moieties.

One embodiment of the present subject matter provides a carbon nanostructure having a substrate with one or more carbon nanotubes situated on a surface of the substrate. A first protective layer may cover portions of the substrate, and a functional second layer may be situated over the first protective layer. This second layer may comprise a bipolar molecule with functional groups or functional moieties.

A further embodiment of the present subject matter provides a method of controlling the density of functional groups or functional moieties on a surface of a carbon nanostructure. The method may include providing a carbon nanostructure having a first protective layer on a surface of the structure and forming a functional second layer over the first protective layer, the second layer having a controllable density of functional groups or functional moieties. The density may be controlled by applying bipolar molecules having a predetermined ratio of functional groups or functional moieties.

An additional embodiment of the present subject matter provides a method of modulating hydrophilicity of a carbon nanostructure. The method may include providing a carbon nanostructure having a first protective layer on a surface of the structure, and forming a hydrophilic second layer over the first protective layer using compounds having one or more —OH groups, —NH₂ groups or —NH— groups.

These embodiments and many other objects and advantages thereof will be readily apparent to one skilled in the art to which the invention pertains from a perusal of the claims, the appended drawings, and the following detailed description of the embodiments.

### BRIEF DESCRIPTION OF THE DRAWINGS

FIG. 1 is a schematic illustration of an exemplary layer-by-layer approach to provide surface functionalization to a CNT and carbon nanostructure.

FIG. 2 is an illustration of a general structure for a molecule with an attached anthraquinone functional moiety for CNT surface functionalization.

FIG. 3 is an illustration of a hydrophilic CNT nanostructure surface with controllable density of anthraquinone moieties.

FIG. 4 is a graphical depiction of a square wave voltammogram overlay of CNT nanostructures functionalized with different ratios of polyoxyethylene alkyl ether anthraquinone 2-carboxylic acid conjugate and C12EG30 for the formation of a second layer, demonstrating the control of functional moiety (anthraquinone) density on functionalized CNT nanostructure surface.

FIG. 5 is a schematic illustration of controlling the number of —OH groups in a bipolar molecule used for the formation of a second layer on a functionalized CNT surface.

FIG. 6 is a schematic illustration of depositing a polyoxyethylene dialkyl ether on a CNT surface to form a second layer on a functionalized CNT surface.

FIG. 7 is an illustration of an exemplary structure of a hydrophilic CNT nanostructure surface and a covalent functionalization of surface —OH groups with an activated anthraquinone ester.

FIG. 8 is a graphical depiction of a square wave voltammogram overlay of differently functionalized CNT nanostructure electrodes.

FIG. 9 is a schematic illustration of an exemplary layer-by-layer introduction of various functional groups onto a CNT nanostructure surface.

FIG. 10 is a graphical depiction of a square wave voltammogram overlay for various embodiments of the present subject matter.

FIG. 11 is a graphical depiction of a square wave voltammogram overlay of a CNT nanostructure electrode functionalized with anthraquinone in buffer solutions at various pHs.

FIG. 12 is a plot of anthraquinone square wave voltammogram redox peak potential versus buffer solution pH for a CNT nanostructure electrode functionalized via an embodiment of the present subject matter.

FIG. 13 is a graphical depiction of an open circuit potential of a CNT nanostructure electrode functionalized using an embodiment of the present subject matter.

FIG. 14 is a plot of open circuit potential versus pH for flowing tap water using an embodiment of the present subject matter.

### DETAILED DESCRIPTION

With reference to the figures, where like elements have been given like numerical designations to facilitate an understanding of the present subject matter, the various embodiments of devices and methods for layer-by-layer carbon nanostructure surface functionalization are described.

While carbon nanotubes (CNTs) are generally regarded as a superior electrode material, grown CNTs are typically hydrophobic. While reference may be made to specific CNTs herein, the claims appended herewith should not be so limited as it is envisioned that embodiments of the present subject matter are applicable to any type CNT such as, but not limited to, single-walled CNTs (SWCNT), multi-walled CNTs (MWCNT), conductive, semi-conductive, or insulated CNTs, and chiral, achiral, open headed, capped, budded, coated, uncoated, functionalized, anchored, or unanchored CNTs, and the like. Hydrophobicity may thus make such CNTs unsuitable for aqueous applications, especially in aqueous solutions with low ionic concentrations. Embodiments of the present subject matter, however, may chemically modify a CNT surface to impart a certain degree of hydrophilicity. Additionally, the use of CNTs as an electrode material is challenging as good contact between the CNT and a conductive surface or electric lead structure must be established. In this regard, CNTs have been used in a composite format with carbon powder on glassy carbon as an electrode material; however, such a mixture of CNT with carbon powder and a composite binder may result in uncertain electrical properties for the CNT.

Embodiments of the present subject matter may grow CNTs on a substrate with an established electric contact. For example, a metal catalyst such as, but not limited to, nickel on top of a titanium adhesion/barrier layer may be deposited on a silicon substrate and annealed at a high temperature to form small catalyst particles. Of course, any type of metal catalyst may be employed in embodiments of the present subject matter and the claims appended herewith should not be limited to the example above. Using chemical vapor deposition techniques, CNTs may grow from the catalyst particles and establish electric contact between the grown CNT and substrate. In co-pending International Application No. PCT/US2010/056350, entitled, “Protection and Surface Modifications of Carbon Nanostructures,” having an international filing date of Nov. 11, 2010, the entirety of which is incorporated herein by reference, the use of an alkyl protective moiety forming an alkyl protective moiety layer to protect the metal catalyst particles (i.e., the electric contact between CNT and substrate) is described. This application generally describes a carbon nanostructure employed as an electrode for the determination of free chlorine and total chlorine concentrations in water.

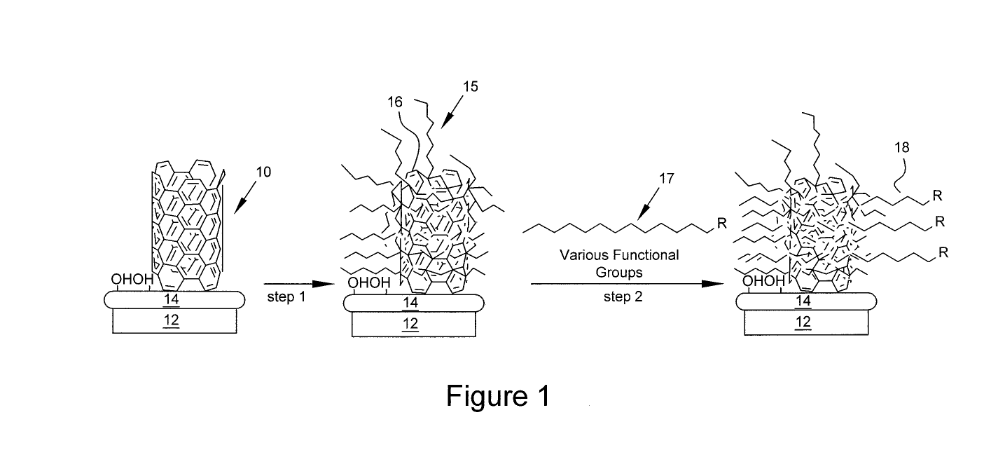

FIG. 1 is a schematic illustration of an exemplary layer-by-layer approach to provide surface functionalization to a CNT and carbon nanostructure. With reference to FIG. 1, to exploit the superior electrical and optical properties of CNTs, embodiments of the present subject matter may covalently attach additional functional groups or functional moieties such as redox mediators and enzyme molecules on top of an exemplary protective layer for the detection of other analytes of interest using a layer-by-layer approach. In step one, grown CNTs 10 on a substrate 12 may be contacted with a composition comprising an alkyl protective moiety under conditions that permit the formation of an alkyl protective layer 15 disposed directly adjacent to at least a portion of the metal catalyst 14 and/or the carbon nanotubes 16. An exemplary alkyl protective moiety may include, but is not limited to, a compound such as an alkane. Non-limiting examples of alkanes include n-octadecane, n-dodecane, eicosane and hexatriacontane. Of course, these examples of alkanes should not limit the scope of the claims appended herewith. The alkyl protective layer 15 may have a thickness in the range of, for example, from about 1 nm to about 500 nm, about 10 nm to about 300 nm, about 50 nm to about 250 nm, or about 50 nm to about 100 nm. At this stage, the CNT surface having the first protective layer 15 may be hydrophobic.

One non-limiting method for the deposition of the first hydrophobic protective layer on an exemplary CNT nanostructure on a substrate may include depositing a solution comprising n-octadecane (10 mM in tetrahydrofuran (THF), 2×5 μL) onto CNTs on a silicon substrate using standard procedures. Upon drying the solvent in air, the treated sample may be placed in a small vial (I.D. ˜2.5 cm) and the vial purged with an Argon stream for 30 seconds and then securely capped. This capped vial may be heated at 120° C. for 16˜24 h, and the sample then cooled to ambient temperature in the capped vial. The sample may then be removed from the vial with forceps and rinsed with THF before drying in air. At this stage, the CNT may be highly hydrophobic with the alkyl protective layer (first layer) in place. Of course, this exemplary method should not limit the scope of the claims appended herewith and is presented simply for representative purposes only.

In step two, other functional groups 17 and functional moieties may then be introduced above this first protective, hydrophobic layer 15, leading to the formation of a second layer 18. One non-limiting method for the second layer functionalization of a CNT nanostructure on a substrate may include providing a CNT nanostructure on a silicon substrate with the first alkyl protective layer in place, followed by depositing a solution of bipolar molecules or a mixture of bipolar molecules with desired functional groups or functional moieties onto the first layer (e.g., 10 mM in THF, 2×5 μL). Upon drying the solvent in air, the treated sample may be placed in a small vial (I.D. ˜2.5 cm) and the vial purged with an Argon stream for 30 seconds and then securely capped. This capped vial may be heated at 80˜120° C. for 16˜24 h, and the sample cooled to ambient temperature in the capped vial. The sample may then be removed from the vial with forceps and rinsed with a solvent to remove excess deposition before drying in air. Again, this exemplary method should not limit the scope of the claims appended herewith and is presently simply for representative purposes only. With the second layer in place, the CNT nanostructure on the substrate may be used as an electrode if no additional functional groups derivatization is required.

In one embodiment, it may be advantageous to use a bipolar molecule (or a mixture of bipolar molecules) where favorable hydrophobic-hydrophobic interaction assists the anchoring of the bipolar molecule onto the first layer 15 with the polar groups exposed for additional manipulation if necessary. An exemplary bipolar molecule may be represented by a compound having the general formula (I):

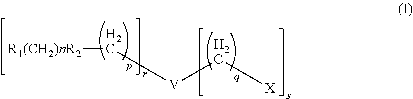

in which:

R₁ represents hydrogen or a C₁₋₅₀ straight or branched alkyl or alkenyl, which is optionally substituted with one or more halogen atoms;

R₂ represents a single bond, an aromatic or alicyclic group, —(OCH₂CH₂)ₘ—, —(OCH₂CH₂CH₂)ₘ—, or —[OCH₂CH(CH₃)]ₘ—, where m and n are each independently 0 to 500;

X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, —NH₂, —NR′R″, —N(COR′)R″, —N⁺R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;

R may be R₁, R₁(CH₂)ₙR₂ or —(CH₂)ₙR₂X;

R′, R″, R′″ may each be independently hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]ₙR;

p, q may each be independently an integral number between 0 and 10;

r, s may each be an integral number between 1 and 4, and 1<r+s<=4; and

V represents a single bond, C, CH, CH₂, Si, N, NH, P, (P═O) or O.

Any polyol may be selected for use as the X substituent in a compound of the formula (I) above. Polyols are compounds having multiple hydroxyl functional groups and may be, for example, diols, triols, tetrols, pentols, and the like. Non-limiting examples of polyols also include polyethylene glycol, pentaerythritol, ethylene glycol, glycerin pentaerythrityl, polyglycerol, sorbitan, polyethylene oxide derivatives of sorbitan, sugar alcohols, trimethylolethane, and trimethylolpropane, among others.

In another embodiment, the bipolar molecule may be represented by a compound having the general formula (II) with two sub-units connected by a linker:

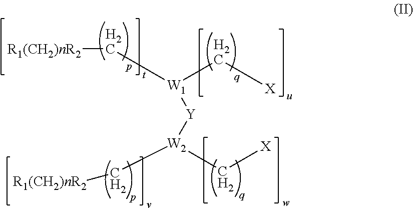

in which:

R₁ represents hydrogen or a C₁₋₅₀ straight or branched alkyl or alkenyl, which is optionally substituted with one or more halogen atoms;

R₂ represents a single bond, an aromatic or alicyclic group, —(OCH₂CH₂)ₘ—, —(OCH₂CH₂CH₂)ₘ—, or —[OCH₂CH(CH₃)]ₘ—, where m and n are each independently 0 to 500;

X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, NH₂, —NR′R″, —N(COR′)R″, —N⁺R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;

R may be R₁, R₁(CH₂)ₙR₂ or —(CH₂)ₙR₂X;

R′, R″, R′″ may each be independently hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]ₙR;

p, q may each be independently an integral number between 0 and 10;

t, v may each be an integral number between 1 and 3, u and w may each be an integral number between 0 and 2, and 1<=t+u<=3, and 1<=v+w<=3;

W₁, W₂ may be independently C, CH, CH₂, Si, N, NH, P, (P═O) or O;

Y represents a single bond or a divalent linker that comprises: C₁₋₅₀ alkyl, alkenyl or aromatic group which is optionally substituted with one or more X; —(OCH₂CH₂)ₘ—, —(OCH₂CH₂CH₂)ₘ—, or —[OCH₂CH(CH₃)]ₘ—, where m and n may each be independently 0 to 500.

Any polyol may be selected for use as the X substituent in a compound of the formula (II) above. Exemplary polyols may be, but are not limited to, diols, triols, tetrols, pentols, polyethylene glycol, pentaerythritol, ethylene glycol, glycerin pentaerythrityl, polyglycerol, sorbitan, polyethylene oxide derivatives of sorbitan, sugar alcohols, trimethylolethane, and trimethylolpropane, and the like.

In a further embodiment, the bipolar molecule may be a compound similar to the compound represented by formula (II) above but may include more than two sub-units connected with multiple linker groups. For example, a bipolar molecule having three sub-units connected with two linker groups in a linear manner may be utilized. It should be appreciated by those skilled in the art that a bipolar molecule with three or more sub-units may be connected with three or more linker groups to form a macro-ring structure as well and such examples should not limit the scope of the claims appended herewith.

Through judicious selection of an exemplary chemical structure of the bipolar molecule, embodiments may introduce an array of functional groups onto a CNT surface above the hydrophobic alkyl protective layer 15. For the CNT nanostructure to be a useful electrode material with long-term stability in aqueous applications, the functionalized CNT surface should, however, be resistant to non-specific adsorption. Additionally, for many surface electrochemical reactions that require participation of H⁺, OH⁻ or H₂O, the functionalized CNT surface should also be highly hydrophilic.

Polyethylene glycol may generally resist non-specific adsorption when deposited on a surface and may render a respective surface hydrophilic to a certain degree. Polyoxyethylene alkyl ethers may also be suitable to be deposited above the hydrophobic alkyl protective layer or first layer 15 on an exemplary CNT to form a hydrophilic polyethylene glycol layer or second layer 18. For example, polyoxyethylene alkyl ethers possess the general formula:

R₃—(OCH₂CH₂)ₘ—OH (III)

in which:

R₃ represents an optionally substituted, linear or branched, saturated or unsaturated, carbo- or heteroalkyl chain bearing 4 to 50 carbon atoms; and

m represents an integer of 1 to 500, and preferably 4 to 200.

Exemplary polyoxyethylene alkyl ethers include, but are not limited to, tetraethyleneglycol monooctyl ether (designated as C8EG4), hexaethyleneglycol monododecyl ether (C12EG6), heptaethyleneglycol monohexadecyl ether (C16EG7) and commercially available detergents, identified by the trade names Brij®30 (C12EG4), Brij®52 (C16EG2), or Brij®56 (C16EG10), Brij®58 (C16EG20), Brij®35 (C12EG30), Brij®78 (C18EG20), Brij®S 100 (C18EG100) (Croda International PLC, East Yorkshire, England).

Embodiments of the present subject matter may employ a myriad of processes to synthesize exemplary bipolar molecules having various functional groups and functional moieties. It should be noted, however, that the subsequent processes detailed below are exemplary only and should not limit the scope of the claims appended herewith. For example, a first process may be used to synthesize N-(6-hydroxy-n-hexyl)p-decylbenzamide represented by the general formula:

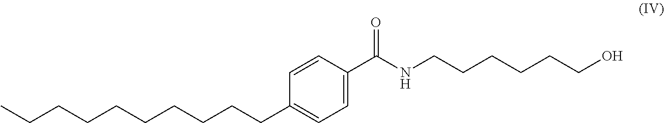

The process may include adding N,N′-dicyclohexylcarbodiimide (DCC, 2.471 g, 11.98 mmol) to a CH₂Cl₂ solution (30 mL) of p-decylbenzoic acid (3.143 g, 11.98 mmol), N-hydroxysuccinimide (NHS, 1.406 g, 12.218 mmol) and triethylamime (Et₃N, 1.67 mL, 11.98 mmol) resulting in a white slurry. After approximately 16 hours at room temperature, the slurry may be filtered using a Buchner filter funnel and rinsed with additional CH₂Cl₂ (30 mL). The filtrate (p-decylbenzoic acid NHS ester) may then be combined and used for subsequent reaction without further purification. A portion of the this filtrate (0.711 mmol) may be mixed with 6-aminohexan-1-ol (0.1755 g, 1.5 mmol) upon stirring. After approximately 2 hours, the reaction mixture may be loaded onto a SiO₂ column and eluted with 5% MeOH in CH₂Cl₂. The fractions containing the desired product may be combined and concentrated to yield a white solid (0.239 g, 93%).

A second process may be used to synthesize N,N′-[2,2′-(ethylenedioxy)bis(ethyl)]di(p-decylbenzamide) represented by the general formula:

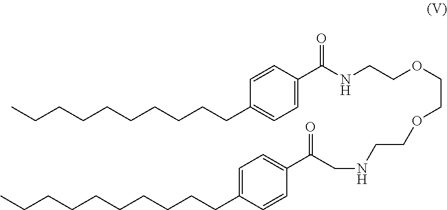

The process may include adding 2,2′-(ethylenedioxy)bis(ethylamine) (0.104 mL, 0.711 mmol) to a CH₂Cl₂ solution (8 mL) of p-decylbenzoic acid NHS ester (1.422 mmol) and Et₃N (0.198 mL, 1.422 mmol). The mixture may be stirred at room temperature for approximately 16 hours before loading onto an SiO₂ column and eluted with 3% MeOH in CH₂Cl₂ and then 5% MeOH in CH₂Cl₂. The fractions containing the desired product may be combined and concentrated to yield a white solid (0.326 g, 72%).

A third process may be used to synthesize N-(11-hydroxy-3,6,9-trioxaundecyl)p-decylbenzamide represented by the general formula:

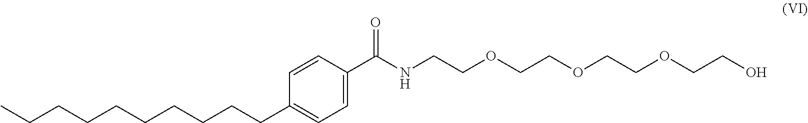

The process may include adding 11-amino-3,6,9-trioxaundecan-1-ol (0.2 g, 1.0 mmol) to a CH₂Cl₂ solution (7 mL) of p-decylbenzoic acid NHS ester (1.0 mmol) and Et₃N (0.14 mL, 1.0 mmol). The mixture may be stirred at room temperature for approximately 1 hour before loading onto an SiO₂ column and eluted with 3% MeOH in CH₂Cl₂. The fractions containing the desired product may be combined and concentrated to yield a solid with a low melting point (0.31 g, 71%).

A fourth process may be used to synthesize N-(11-hydroxy-3,6,9-trioxaundecyl)octadecanamide represented by the general formula:

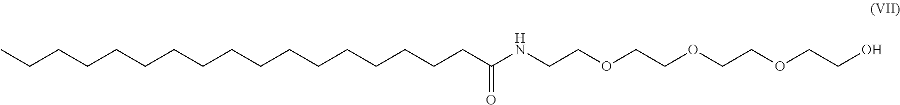

The process may include adding DCC (2.512 g, 12.173 mmol) to a CH₂Cl₂ solution (30 mL) of stearic acid (3.463 g, 12.173 mmol), NHS (1.429 g, 12.416 mmol) and Et₃N (1.7 mL, 12.173 mmol) resulting in an opaque solution, which may slowly turn into a white slurry. After approximately 16 hours at room temperature, a white solid may be filtered using a Buchner filter funnel and rinsed with additional CH₂Cl₂ (30 mL) whereby the filtrate (stearic acid NHS ester) may be combined and used for subsequent reaction without further purification. A portion of the filtrate (2.815 mmol) may be mixed with 11-amino-3,6,9-trioxaundecan-1-ol (0.483 g, 2.5 mmol) and Et₃N (0.39 mL, 2.82 mmol) upon stirring. The mixture may then be concentrated to −5 mL and then loaded onto an SiO₂ column and eluted with 3% MeOH in CH₂Cl₂. The fractions containing the desired product may be combined and concentrated to yield a white solid (0.772 g, 72%).

A fifth process may be used to synthesize C16EG100CH₃ represented by the general formula:

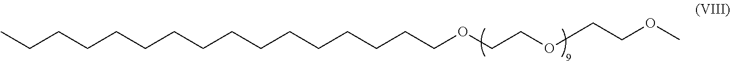

The process may include dissolving waxy solid Brij®56 (a mixture designated as C16EG10) (1.808 g, 2.65 mmol) in anhydrous DMF (6 mL). Upon the addition of NaH (57% oil dispersion, 0.223 g, 5.29 mmol), the mixture may turn slightly foamy with gas evolution. After introduction of CH₃I (0.66 mL, 10.6 mmol), the reaction may become warm and gas evolution subside. After approximately 5 hours, the solvent may be removed in vacuo and the resultant white residue suspended in CH₂Cl₂ (2 mL) and then loaded onto an SiO₂ column and eluted with 3% MeOH in CH₂Cl₂ and then 5% MeOH in CH₂Cl₂. After removal of the solvent, the desired product may be obtained as a white waxy solid (0.963 g, 53% yield).

A sixth process may be used to synthesize C16EG10C6 represented by the general formula:

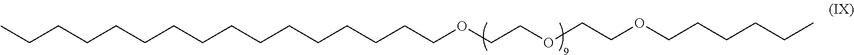

The process may include dissolving waxy solid Brij®56 (C16EG10) (3.53 g, 5.17 mmol) in anhydrous DMF (10 mL), followed by adding NaH (57% oil dispersion, 1.088 g, 25.84 mmol). 1-bromohexane (4.354 mL, 31.02 mmol) may then be introduced to the mixture resulting in a slurry which may be stirred at room temperature in a sealed flask after gas evolution subsides. After approximately 16 hours, the solvent may be removed in vacuo and the resultant white residue mixed with an ethyl acetate/hexanes mixture solvent (EtOAc/hex, 1:1 v/v, ˜5 mL). This mixture may then be loaded onto an SiO₂ column and eluted with EtOAc/hex (1:1 v/v) and then 3% MeOH in CH₂Cl₂. The fractions containing the product may be combined to yield a waxy solid (˜3.15 g) and may then be subjected to a second SiO₂ column and eluted with 3% MeOH in CH₂Cl₂ to yield a white waxy solid (2.872 g, 72% yield).

A seventh process may be used to synthesize heptaethylene glycol dihexadecyl ether (C16EG7C16) represented by the general formula:

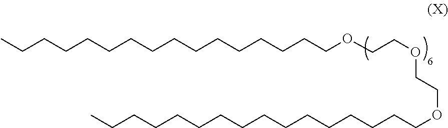

The process may include dissolving waxy solid heptaethylene glycol monohexadecyl ether (pure compound from Sigma-Aldrich, designated as C16EG7) (0.11 g, 0.2 mmol) in anhydrous DMF with NaH (57% oil dispersion, 42 mg, 1.0 mmol), followed by the addition of 1-bromohexadecane (0.366 g, 1.2 mmol). The resulting mixture may be stirred at room temperature for approximately 16 hours in a sealed flask before removal of the solvent in vacuo and suspension of the resultant white residue in an ethyl acetate/hexanes mixture solvent (EtOAc/hex, 1:1 v/v, ˜2 mL). The suspension may then be loaded onto an SiO₂ column and eluted with EtOAc/hex (1:1 v/v) and then 3% MeOH in CH₂Cl₂ to afford the desired product as a waxy film (0.147 g, 95% yield).

An eighth process may be used to synthesize C18EG20C16 represented by the general formula:

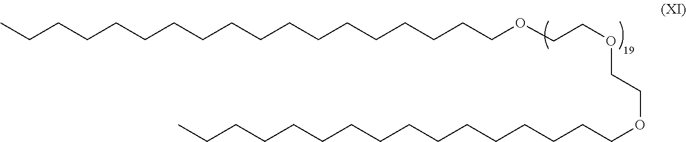

The process may include mixing waxy solid Brij®78 (a mixture designated as C18EG20) (2.302 g, 2.0 mmol) with anhydrous DMF (10 mL), followed by the addition of NaH (57% oil dispersion, 0.21 g, 5.0 mmol). The mixture may be heated whereby gas evolution may commence and the mixture may become free-flowing. After approximately 15 minutes, 1-bromohexadecane (1.832 g, 6.0 mmol) may be introduced and the resulting slurry stirred at room temperature in a sealed flask. After approximately 16 hours, the solvent may be removed in vacuo and the white residue mixed with 3% MeOH in CH₂Cl₂ (5 mL) and SiO₂ (2 g). The slurry may then be loaded onto an SiO₂ column, and eluted with 3% MeOH in CH₂Cl₂ and then 5% MeOH in CH₂Cl₂. Less polar fractions of the product may be discarded, and the more polar fractions of product may be combined and concentrated in vacuo to afford a white waxy solid (1.619 g, 59%).

A ninth process may be used to synthesize C12EG30C12 represented by the general formula:

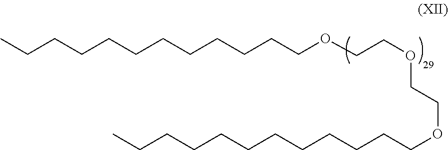

The process may include mixing white solid Brij®35 (a mixture designated as C12EG30) (2.624 g, 1.741 mmol) with anhydrous DMF (10 mL), followed by adding NaH (57% oil dispersion, 0.183 g, 4.35 mmol). The mixture may be heated whereby gas evolution may commence and the mixture may become free-flowing. After approximately 15 minutes, 1-bromododecane (1.252 mL, 5.223 mmol) may be introduced and the resulting slurry stirred at room temperature in a sealed flask. After approximately 16 hours, the solvent may be removed in vacuo and the white residue was mixed with 3% MeOH in CH₂Cl₂ (5 mL) and SiO₂ (2 g). The slurry may then be loaded onto an SiO₂ column, and eluted with 1% MeOH in CH₂Cl₂, 5% MeOH in CH₂Cl₂ and then 8% MeOH in CH₂Cl₂. The fractions containing the desired product may be combined and concentrated in vacuo to afford a white solid (2.91 g, 100%).

A tenth process may be used to synthesize C16EG9CH₂CH₂N3 represented by the general formula:

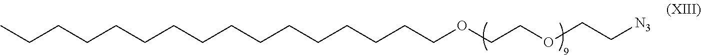

The process may include dissolving waxy solid Brij®56 (C16EG10) (6.20 g, 9.078 mmol) in anhydrous THF (30 rnL), followed by adding Et₃N (1.9 mL, 13.62 mmol) thereto. Toluenesulfonyl chloride (1.904 g, 10.0 mmol) may be introduced to the mixture resulting in a slurry which may be stirred at room temperature in a sealed flask. After approximately 3 days, a solid may be filtered using a Buchner filter funnel and the filtrate concentrated to afford a milky liquid (8.16 g). Anhydrous DMF (10 mL) and NaN₃ (0.649 g, 10.0 mmol) may be mixed with the milky liquid and then stirred at 80° C. in a sealed flask for approximately 24 hours. The solvent may then be removed in vacuo and the residue mixed with EtOAc/hex (1:1 v/v) (˜10 mL) and SiO₂ (5 g). The resulting slurry may be loaded onto an SiO₂ column and eluted with EtOAc/hex (1:1 v/v), 3% MeOH in CH₂Cl₂ and then 5% MeOH in CH₂Cl². Fractions containing the desired product may be combined to afford a light yellow waxy solid (5.3 g, 82%).

An eleventh process may be used to synthesize C16EG9CH₂CH₂NH₂ represented by the general formula:

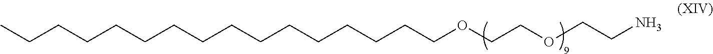

The process may include dissolving a light yellow waxy solid (a mixture designated as C16EG9CH₂CH₂N3) (1.87 g, ˜2.63 mmol) in THF (20 mL), followed by adding a Raney Ni suspension (50% slurry in H₂O, ˜1 mL). Upon gas evolution subsiding, the solid may be filtered using glass wool in a pipette and rinsed with THF. The filtrate may then be concentrated and loaded onto an SiO₂ column and eluted with 5% MeOH in CH₂Cl₂, then 10% MeOH in CH₂Cl₂ and then MeOH/CH₂Cl₂/saturated NH₃ aqueous solution (1:5:0.1 v/v/v). The desired product may be obtained as a white solid (1.006 g, 56%).

A twelfth process may be used to synthesize (C16EG9CH₂CH₂S)₂ represented by the general formula:

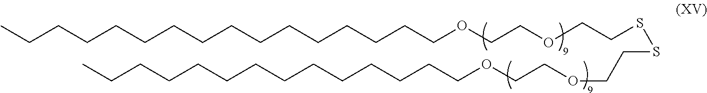

The process may include dissolving waxy solid Brij®56 (C16EG10) (1.282 g, 1.877 mmol) in anhydrous CH₂Cl₂ (10 mL), followed by adding Et₃N (0.53 mL, 3.75 mmol) thereto. Methanesulfonyl chloride (0.22 mL, 2.82 mmol) may be introduced to the mixture at 0° C. resulting in a suspension which may be stirred at room temperature in a sealed flask for approximately 30 min. This reaction mixture may then be loaded onto an SiO₂ column and eluted with 3% MeOH in CH₂Cl₂ and then 10% MeOH in CH₂Cl₂. The fractions may be combined and concentrated to afford a waxy solid (1.27 g), which may be mixed with anhydrous DMF (5 mL) and KSAc (0.381 g, 3.338 mmol). This mixture may be stirred at 80° C. in a sealed flask for approximately 24 hours resulting in a gel-like suspension. The suspension may be cooled to room temperature and mixed with 5% MeOH in CH₂Cl₂ (5 mL) and SiO₂ (5 g) and then loaded onto an SiO₂ column and eluted with 5% MeOH in CH₂Cl₂ and then 10% MeOH in CH₂Cl₂. Fractions containing the product may be combined and concentrated into a red oil, and the red oil subjected to a second SiO₂ column and eluted with 5% MeOH in CH₂Cl₂ and then 10% MeOH in CH₂Cl₂ to afford a pale yellow waxy solid (1.186 g). This solid may then be dissolved in MeOH (5 mL) and then treated with NaOH (0.134 g, 3.34 mmol), stirred at room temperature in a sealed flask for approximately 16 hours, and then stirred in open air for approximately 16 hours to oxidize any free thiol —SH to its corresponding disulfide. The resulting solid may then be mixed with 3% MeOH in CH₂Cl₂ (3 mL) and SiO₂ (2 g) resulting in a slurry. This slurry may be loaded onto a SiO₂ column and eluted with 3% MeOH in CH₂Cl₂ and then 10% MeOH in CH₂Cl₂. The product may be obtained as a pale yellow solid (1.04 g, 79% yield).

A thirteenth process may be used to synthesize C16EG10SO₃⁻ represented by the general formula:

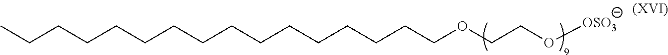

The process may include mixing solid Brij®56 (C16EG10) (0.8829 g, 1.293 mmol) with a solid sulfur trioxide trimethylamine complex (SO₃.NMe₃, 0.201 g, 1.44 mmol) in a sealed flask under Argon. The mixture may then be warmed at 90° C. for approximately 16 hours resulting in a white slurry which slowly turns into a clear oil, indicating the consumption of SO₃.NMe₃. The oil may then turn into a white solid upon cooling. As the solid is not soluble in THF, this indicates conversion of C16EG10 to its sulfate C16EG10SO₃⁻. The resultant solid is soluble in a THF/MeOH (1:1 v/v) mixture solvent and may be employed for CNT nanostructure surface functionalization without further purification.

A fourteenth process may be used to synthesize C18EG20SO₃⁻ represented by the general formula:

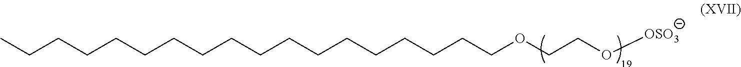

The process may include mixing Brij®78 (C18EG20) (1.331 g, 1.157 mmol) with a solid sulfur trioxide trimethylamine complex (SO₃.NMe₃, 0.177 g, 1.272 mmol) in a sealed flask under Argon. The mixture may then be warmed at 90° C. for approximately 16 hours turning into a clear oil which indicates the consumption of SO₃.NMe₃. The oil may then turn into a white solid upon cooling which is soluble in a THF/MeOH (1:1 v/v) mixture solvent for CNT nanostructure surface functionalization.

A fifteenth process may be used to synthesize C12EG30SO₃⁻ represented by the general formula:

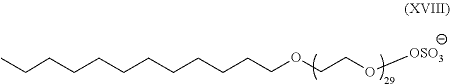

The process may include mixing Brij®35 (C12EG30) (1.574 g, 1.044 mmol) with a solid sulfur trioxide trimethylamine complex (SO₃.NMe₃, 0.16 g, 1.149 mmol) in a sealed flask under Argon. The mixture may then be warmed at 90° C. for approximately 16 hours turning into a clear oil which indicates the consumption of SO₃.NMe₃. The oil may then turn into a white waxy solid upon cooling which is soluble in a THF/MeOH (1:1 v/v) mixture solvent for CNT nanostructure surface functionalization.

A sixteenth process may be used to synthesize C16EG7SO₃⁻ represented by the general formula:

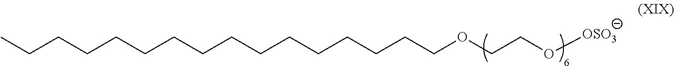

The process may include mixing solid heptaethylene glycol monohexadecyl ether (C16EG7, 0.712 g, 1.293 mmol) with a solid sulfur trioxide trimethylamine complex (SO₃.NMe₃, 0.198 g, 1.42 mmol) in a sealed flask under Argon. The mixture may then be warmed at 90° C. for approximately 16 hours turning into a clear oil which indicates the consumption of SO₃.NMe₃. The oil may then turn into a white waxy solid upon cooling which is soluble in a THF/MeOH (1:1 v/v) mixture solvent for CNT nanostructure surface functionalization.

A seventeenth process may be used to synthesize C8EG3CH₂CH₂N₃ represented by the general formula:

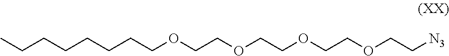

The process may include adding toluenesulfonyl chloride (0.482 g, 2.53 mmol) to a THF solution (8 mL) of tetraethylene glycol monooctyl ether (pure compound from Sigma-Aldrich designated as C8EG4) (0.646 g, 2.11 mmol) and Et₃N (0.593 mL, 4.22 mmol) resulting in a slurry. This slurry may be stirred in a sealed flask at room temperature for approximately 24 hours whereby an additional 0.2 eq of toluenesulfonyl chloride may be introduced followed by additional stirring at 40° C. for approximately 16 hours. The solvent may then be removed, and the residue loaded onto an SiO₂ column and eluted with EtOAc/hex 1:2 then 1:1 to yield an oil (0.865 g, 89%). This oil (tetraethylene glycol monooctyl ether tosylate) (0.216 g, 0.469 mmol) may be mixed with anhydrous DMF (5 mL) and NaN₃ (46 mg, 0.704 mmol) and then stirred at 85° C. in a sealed flask for approximately 24 hours. The solvent may be removed in vacuo and the residue mixed with EtOAc/hex (1:2 v/v) (˜2 mL) resulting in a slurry. This slurry may be loaded onto an SiO₂ column and eluted with EtOAc/hex (1:1 v/v) to afford the desired product as a clear oil (0.156 g, 100%).

An eighteenth process may be used to synthesize C8EG3CH₂CH₂NH₂ represented by the general formula:

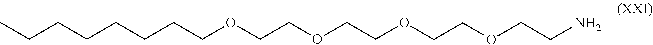

The process may include mixing an oil C8EG3CH₂CH₂N₃ (0.156 g, 0.469 mmol) with THF (3 mL), H₂O (20 μL) and triphenyl phosphine (0.185 g, 0.704 mmol). The resulting mixture may then be stirred at room temperature under Argon in a sealed flask for approximately 16 hours. The solvent may be removed and residue loaded onto an SiO₂ column and eluted with 10% MeOH in CH₂Cl₂ and then 10% MeOH in CH₂Cl₂ with a 1% saturated NH₃ aqueous solution to afford the desired product as a clear film (0.11 g, 77% yield).

A nineteenth process may be used to synthesize 12-(n-octyl)-12-aza-3,6,9-trioxa-1-eicosanol represented by the general formula:

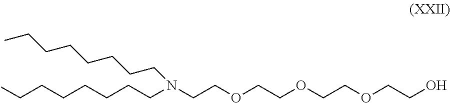

The process may include mixing dioctylamine (0.71 mL, 2.37 mmol) with tetraethylene glycol monotosylate (0.412 g, 1.18 mmol) in a sealed flask. The mixture may be stirred and warmed at 80° C. for approximately 16 hours resulting in a slurry. Upon cooling, the slurry may be suspended in CH₂Cl₂ (5 mL), followed by adding Et₃N (0.164 mL, 1.18 mmol) and acetic anhydride (0.112 mL, 1.18 mmol) at 0° C. After approximately 30 min, the reaction mixture may be diluted with MeOH (1.0 mL) and then concentrated whereby the residue may be loaded onto an SiO₂ column and eluted with 5% MeOH in CH₂Cl₂ and then 10% MeOH in CH₂Cl₂ to yield the desired product as a clear oil (0.402 g, 82% yield).

A twentieth process may be used to synthesize N,N-di-(n-octyl)-N′-(11-hydroxy-3,6,9-trioxaundecyl)succinamide represented by the general formula:

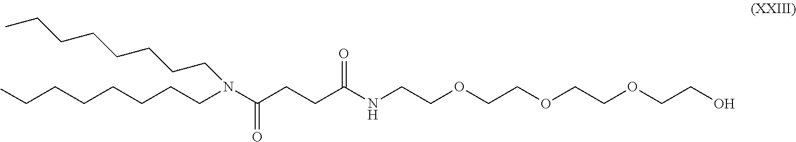

The process may include mixing dioctylamine (0.302 mL, 1.0 mmol) with succinic anhydride (0.11 g, 1.1 mmol) and diisopropylethylamine (DIPEA, 0.348 mL, 2.0 mmol) in CH₂Cl₂ (2 mL). After approximately 16 hours, the solution may be treated with 11-amino-3,6,9-trioxaundecan-1-ol (0.193 g, 1.0 mmol) and 3-diethoxyphosphoryloxy-1,2,3-benzotriazin-4(3H)-one (0.329 g, 1.1 mmol) resulting in a light yellow solution. This solution may be stirred at room temperature for approximately 4 hours before quenching with ethylene diamine (0.1 mL) to form a yellow suspension. The suspension may be loaded onto an SiO₂ column and eluted with 5% MeOH in CH₂Cl₂ to afford the desired product as a clear oil (0.306 g, 59% yield over two steps).

A twenty first process may be used to synthesize N,N-di-(n-octadecyl)-N′-(11-hydroxy-3,6,9-trioxaundecyl)succinamide represented by the general formula:

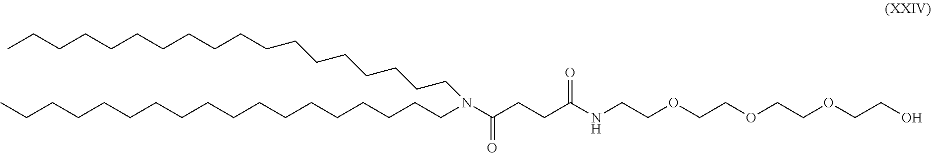

The process may include mixing dioctadecylamine (0.261 g, 0.5 mmol) with succinic anhydride (0.055 g, 0.55 mmol) and diisopropylethylamine (DIPEA, 0.174 mL, 1.0 mmol) in CH₂Cl₂ (1 mL). After approximately 16 hours, the solution may be treated with 11-amino-3,6,9-trioxaundecan-1-ol (0.0966 g, 0.5 mmol) and 3-diethoxyphosphoryloxy-1,2,3-benzotriazin-4(3H)-one (0.165 g, 0.55 mmol) resulting in a light yellow solution. This solution may be stirred at room temperature for approximately 4 hours before quenching with ethylene diamine (0.15 mL) to form a yellow suspension. The suspension may be loaded onto a SiO₂ column and eluted with 3% MeOH in CH₂Cl₂ to afford the desired product as a clear oil (0.355 g, 89% yield over two steps).

A twenty second process may be used to synthesize N,N-di-(n-octadecyl)-N′-(6-hydroxyhexyl)succinamide represented by the general formula:

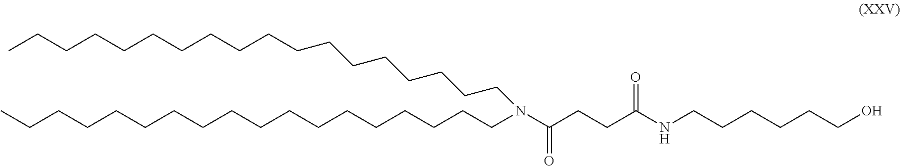

The process may include mixing dioctadecylamine (0.261 g, 0.5 mmol) with succinic anhydride (0.055 g, 0.55 mmol) and diisopropylethylamine (DIPEA, 0.174 mL, 1.0 mmol) in CH₂Cl₂ (1 mL). After approximately 16 hours, the solution may be treated with 6-amino-hexan-1-ol (0.0585 g, 0.5 mmol) and 3-diethoxyphosphoryloxy-1,2,3-benzotriazin-4(3H)-one (0.165 g, 0.55 mmol) resulting in a light yellow solution. This solution may be stirred at room temperature for 16 hours before quenching with ethylene diamine (0.15 mL) to form a yellow suspension. The suspension may be loaded onto a SiO₂ column and eluted with 3% MeOH in CH₂Cl₂ to afford the desired product as a clear oil (0.305 g, 86% yield over two steps).

A twenty third process may be used to synthesize 12-(n-octadecyl)-12-aza-3,6,9-trioxa-1-triacontanol represented by the general formula:

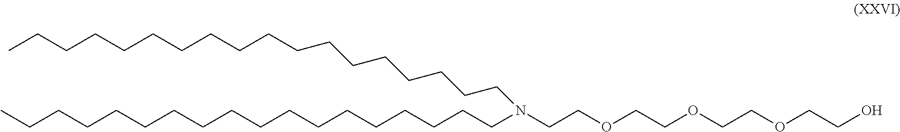

The process may include mixing solid dioctadecylamine (0.367 g, 0.703 mmol) with tetraethylene glycol monotosylate (0.223 g, 0.639 mmol) in a sealed flask. The mixture may then be stirred at 90° C. for approximately 16 hours to form an amber oil. Upon cooling, the resultant yellow solid may be suspended in 3% MeOH in CH₂Cl₂ (5 mL) followed by adding Et₃N (0.21 mL, 1.5 mmol) and acetic anhydride (0.0354 mL, 0.375 mmol) resulting in a clearly slurry. After approximately 30 min, the reaction mixture may be quenched with ethylenediamine (0.15 mL) and loaded onto an SiO₂ column and eluted with 3% MeOH in CH₂Cl₂, 5% MeOH in CH₂Cl₂ and then 10% MeOH in CH₂Cl₂ to yield the desired product as a waxy solid (0.212 g, 48% yield).

A twenty fourth process may be used to synthesize 3α,7α,12α-trihydroxy-5β-cholan-24-oic acid N,N-di-(n-octadecyl)amide represented by the general formula:

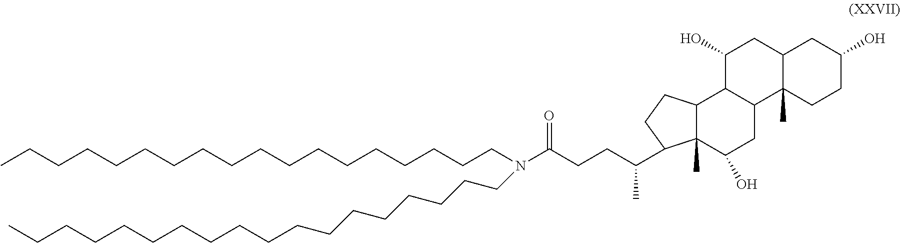

The process may include introducing diisopropylethylamine (0.082 mL, 0.472 mmol) and 3-diethoxyphosphoryloxy-1,2,3-benzotriazin-4(3H)-one (77.7 mg, 0.26 mmol) to a slurry of 3α,7α,12α-trihydroxy-5β-cholan-24-oic acid (96.5 mg, 0.236 mmol) and dioctadecylamine (123.3 mg, 0.236 mmol) in CH₂Cl₂ (5 mL) turning the slurry into a clear yellow solution after approximately 16 hours of stirring. Ethylenediamine (0.025 mL) may then be added to the slurry and mixed with SiO₂ (1 g) and then loaded onto a SiO₂ column and eluted with 5% MeOH in CH₂Cl₂ to yield the desired product as a white solid (0.186 g, 86% yield).

A twenty fifth process may be used to synthesize (2,2,2-trimethylol) azidoethane tri(3,6,9,12-tetraoxaeicosanyl)ether represented by the general formula:

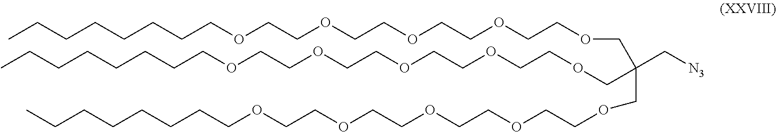

The process may include stirring (2-bromomethyl)-(2-hydroxymethyl)-1,3-propanediol (2.5747 g, 12.93 mmol) with NaN₃ (1.163 g, 17.9 mmol) in anhydrous DMF (10 mL) in a sealed flask at 85° C. for approximately three days. The solvent may be removed in vacuo, and the resulting white slurry purified by loading onto an SiO₂ column and eluted with 10% MeOH in CH₂Cl₂ and then 20% MeOH in CH₂Cl₂ to yield the desired product (2-azidomethyl)-(2-hydroxymethyl)-1,3-propanediol as a white soft solid upon standing (2.059 g, 99% yield). (2-azidomethyl)-(2-hydroxymethyl)-1,3-propanediol (57.4 mg, 0.357 mmol) may then be mixed with NaH (57% oil dispersion, 68 mg, 1.61 mmol) in anhydrous DMF (5 mL). Tetraethylene glycol monooctyl ether tosylate (0.525 g, 1.14 mmol) may then be introduced into the mixture resulting in a slurry. The slurry may then be stirred in a sealed flask at room temperature for approximately 24 hours, and additional NaH (57% oil dispersion, 42 mg) introduced followed by the addition of tetraethylene glycol monooctyl ether tosylate (0.10 g). This reaction mixture may then be stirred at room temperature for approximately three days whereupon the solvent may be removed and residue loaded onto an SiO₂ column and eluted with EtOAc/hex (1:2 v/v) then 5% MeOH in CH₂Cl₂ to yield the desired product (2,2,2-trimethylol) azidoethane tri(3,6,9,12 tetraoxaeicosanyl)ether as a clear oil (0.37 g).

A twenty sixth process may be used to synthesize (2,2,2-trimethylol) ethylamine tri(3,6,9,12-tetraoxaeicosanyl)ether represented by the general formula:

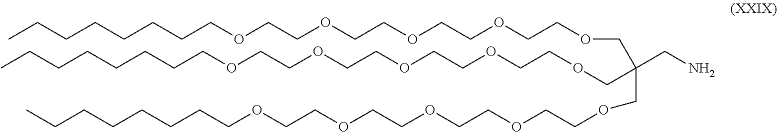

The process may include subjecting the clear oil (2,2,2-trimethylol) azidoethane tri(3,6,9,12-tetraoxaeicosanyl)ether (0.37 g, ˜0.357 mmol) to reduction with triphenyl phosphine (0.14 g, 0.536 mmol) in THF (3 mL) with H₂O (10 mg) in a sealed flask under Argon upon stirring for approximately 24 hours. The solvent may then be removed and the residue mixed with CH₂Cl₂ (1 mL) and then loaded onto an SiO₂ column and eluted with 5% MeOH in CH₂Cl₂, and then a mixture solvent of MeOH/CH₂Cl₂/saturated aqueous ammonia (10:90:1 v/v/v) to afford the desired product (2,2,2-trimethylol) ethylamine tri(3,6,9,12-tetraoxaeicosanyl)ether as a clear oil (0.26 g, 73% yield).

A twenty seventh process may be used to synthesize C12EG29CH₂CH₂NHCH₂CH₂OH represented by the general formula:

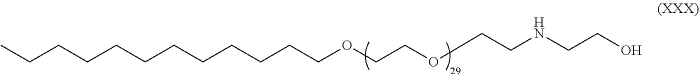

The process may include mixing pellets of Brij®35 (C12EG30) (9.042 g, 6.0 mmol) with Et₃N (1.254 mL, 9.0 mmol) in THF (4 mL). The mixture may be heated to a clear solution and toluenesulfonyl chloride (1.258 g, 6.6 mmol) introduced thereto resulting in a milky slurry. The slurry may be stirred at room temperature for approximately three days and a solid filtered using glass wool in a glass pipette. The solid may then be rinsed with THF (˜10 mL) and the filtrate concentrated to a viscous oil in vacuo which may then be mixed with ethanolamine (3.62 mL, 60 mmol) in a sealed flask upon stirring at 90° C. for approximately 16 hours resulting in a slightly yellow reaction mixture. Upon cooling the mixture may become a waxy solid whereupon the solid may be dissolved in 5% MeOH in CH₂Cl₂, loaded onto an SiO₂ column and eluted with 5% MeOH in CH₂Cl₂, 10% MeOH in CH₂Cl₂, and then a mixture solvent of MeOH/CH₂Cl₂/saturated aqueous ammonia (10:90:1 followed by 20:80:2 v/v/v) to yield the desired product as a slightly yellow waxy solid (6.408 g, 69%).

A twenty eighth process may be used to synthesize N—(C16EG9CH₂CH₂) (±)-α-lipoic acid amide represented by the general formula:

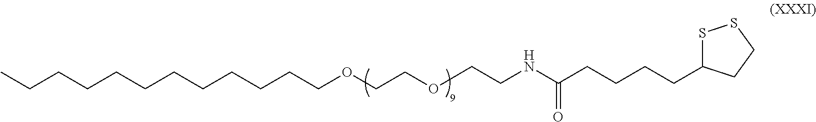

The process may include introducing C16EG9CH₂CH₂NH₂ (0.1076 g, 0.1577 mmol) to a solution of (±)-α-lipoic acid (39 mg, 0.189 mmol) and diisopropylethylamine (55 μL, 0.315 mmol) in CH₂Cl₂ (2 mL), followed by adding 3-diethoxyphosphoryloxy-1,2,3-benzotriazin-4(3H)-one (56.6 mg, 0.189 mmol) resulting in a yellow solution. After approximately 16 hours, ethylenediamine (15 μL) may be added to the solution resulting in a slurry which may be loaded onto an SiO₂ column and eluted with 3% MeOH in CH₂Cl₂, 5% MeOH in CH₂Cl₂ and then 10% MeOH in CH₂Cl₂ to afford the desired product as a light yellow waxy solid (0.103 g, 75% yield).

A twenty ninth process may be used to synthesize N—(C16EG9CH₂CH₂) anthraquinone-2-carboxylic acid amide represented by the general formula:

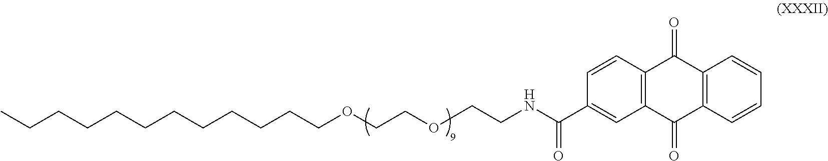

The process may include introducing C16EG9CH₂CH₂NH₂ (0.101 g, 0.149 mmol) to a solution of anthraquinone-2-carboxylic acid (45 mg, 0.178 mmol) and diisopropylethylamine (52 μL, 0.297 mmol) in CH₂Cl₂ (2 mL), followed by adding 3-diethoxyphosphoryloxy-1,2,3-benzotriazin-4(3H)-one (53.4 mg, 0.178 mmol) resulting in a yellow solution. After approximately 3 hours, ethylenediamine (10 μL) may be added to the solution resulting in a slurry which may be loaded onto an SiO₂ column and eluted with 3% MeOH in CH₂Cl₂, 5% MeOH in CH₂Cl₂ and then 10% MeOH in CH₂Cl₂ to afford the desired product as a light yellow waxy solid (93 mg, 68% yield).

A thirtieth process may be used to synthesize N—(C12EG29CH₂CH₂)—N-(2-hydroxyethyl)anthraquinone-2-carboxylic acid amide represented by the general formula:

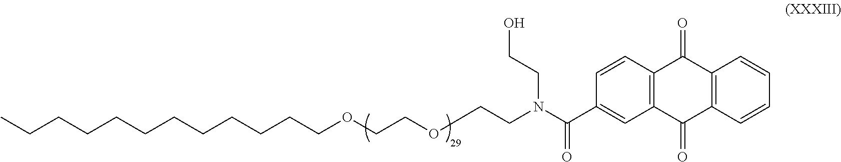

The process may include introducing C12EG29CH₂CH₂NHCH₂CH₂OH (0.203 g, 0.131 mmol) to a solution of anthraquinone-2-carboxylic acid (33 mg, 0.131 mmol) and diisopropylethylamine (46 μL, 0.262 mmol) in CH₂Cl₂ (1 mL), followed by adding 3-diethoxyphosphoryloxy-1,2,3-benzotriazin-4(3H)-one (39.2 mg, 0.131 mmol) resulting in a yellow solution. After approximately 16 hours, ethylenediamine (10 μL) may be added to the solution resulting in a slurry which may be loaded onto an SiO₂ column and eluted with 5% MeOH in CH₂Cl₂ and then 10% MeOH in CH₂Cl₂ to afford the desired product as a light yellow waxy solid (172 mg, 75% yield).

A thirty first process may be used to synthesize N—(C12EG29CH₂CH₂)—N-(2-hydroxyethyl)3-(2,5-dimethoxyphenyl)propionic acid amide represented by the general formula:

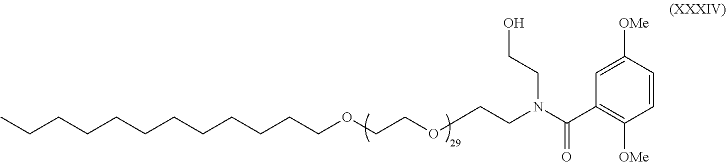

The process may include introducing C12EG29CH₂CH₂NHCH₂CH₂OH (0.576 g, 0.371 mmol) to a solution of 3-(2,5dimethoxyphenyl)propionic acid (78.08 mg, 0.371 mmol) and diisopropylethylamine (129 μL, 0.742 mmol) in CH₂Cl₂ (4 mL), followed by adding 3-diethoxyphosphoryloxy-1,2,3-benzotriazin-4(3H)-one (111 mg, 0.371 mmol) resulting in a yellow solution. After approximately 16 hours, ethylenediamine (30 μL) may be added to the solution resulting in a slurry which may be loaded onto an SiO₂ column and eluted with 5% MeOH in CH₂Cl₂ and then 10% MeOH in CH₂Cl₂ to afford the desired product as a light yellow waxy solid (0.42 g, 65% yield).

With continued reference to FIG. 1 and the aforementioned examples, an exemplary CNT nanostructure functionalized using a layer-by-layer approach, i.e., forming a first protective (e.g., alkyl) layer followed by a second layer of polyoxyethylene alkyl ether or other layer. These embodiments may be employed as an electrode for free chlorine concentration determination in tap water, as a potentiometric pH sensor, an amperometric pH sensor, a biometric sensor or electrode, or a voltammetric pH sensor or other sensor or electrode.

It should be noted that polyoxyethylene alkyl ethers may be derivatized to form bipolar molecules with additional functionalities. For example, the —OH group in polyoxyethylene alkyl ethers may react with SO₃ or P₂O₅ to create a bipolar molecule which can be used to introduce —OSO₃⁻ or —OPO₃H₂ groups onto an exemplary CNT electrode surface. It should also be noted that an —OH group can form esters with various carboxylic acids and may undergo a variety of transformations to be replaced by groups such as, but not limited to, —N₃, —NH₂ and —SH or —S—S—, to name a few.

Additionally, in one embodiment polyoxyethylene alkyl ether may be converted to a respective mesylate or tosylate, which could then be substituted with nucleophilic groups including, but not limited to, halide, azide, sulfide or masked thiol such as thioacetate, NH₃, primary amine, secondary amine and tertiary amine. In yet another embodiment, polyoxyethylene alkyl ether may react in the presence of NaH with activated acetate such as tert-butyl bromoacetate followed by deprotection of tert-butyl ester to yield polyoxyethylene alkyl ether with a terminal —COOH group. Another embodiment may employ polyoxyethylene alkyl ether with a terminal —NH₂ group to react with succinic anhydride to introduce a terminal —COOH group.

A terminal —NH₂ or —NH— group in derivatized polyoxyethylene alkyl ether may react with various carboxylic acids via an amide bond formation. Thus, a range of exemplary redox mediator moieties may be covalently linked to polyoxyethylene alkyl ether. Exemplary, non-limiting mediators include anthraquinone 2-carboxylic acid, 3-(2,5-dimethoxyphenyl)propionic acid and (±)-α-lipoic acid. In the case of 3-(2,5-dimethoxyphenyl)propionic acid, the hydroquinone moiety may be protected with methyl groups and hence the hydroquinone/benzoquinone redox pair would not be present after the second layer deposition. After a few scans of cyclic voltammetry, the 2,5-dimethoxyphenyl moiety may then be oxidized to generate a desired hydroquinone/benzoquinone redox pair for electrochemical sensing of solution pH. It should be noted that many other masked/protected functional groups or functional moieties may be unmasked/deprotected electrochemically once they are introduced onto an exemplary CNT electrode surface, thus, such examples should not limit the scope of the claims appended herewith.

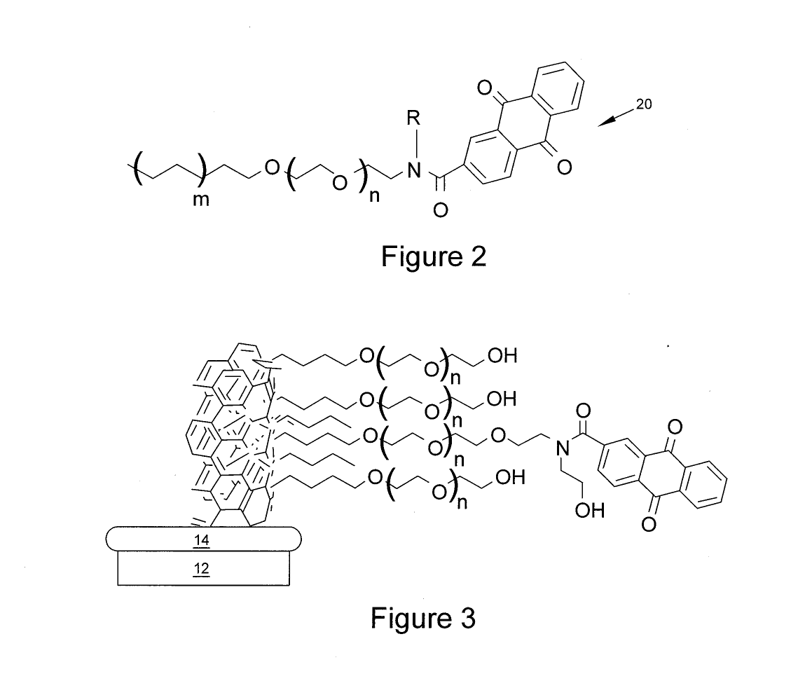

FIG. 2 is an illustration of a general structure for a molecule with an attached anthraquinone functional moiety for CNT surface functionalization. With reference to FIG. 2, a molecule 20 is provided having a polyoxyethylene alkyl ether covalently attached with redox mediators [e.g., anthraquinone (AQ)] or another functional group (e.g., polyoxyethylene alkyl ether conjugate) to form an exemplary second layer 18 above the first protective layer 15 on a CNT structure (see FIG. 1). Peak potential of the respective redox mediators may be used to determine solution pH according to the Nernst Equation.

FIG. 3 is an illustration of a hydrophilic CNT nanostructure surface with controllable density of anthraquinone moieties. With reference to FIG. 3, the density of exemplary functional groups may be, in one embodiment, controlled by mixing polyoxyethylene alkyl ether containing a first functional group with polyoxyethylene alkyl ether containing a second functional group. For example, polyoxyethylene alkyl ether derivatized with a terminal —NH₂ or —NH— group can be mixed with a non-derivatized polyoxyethylene alkyl ether to control the density of the surface —NH₂ or —NH— group. Similarly, polyoxyethylene alkyl ether anthraquinone 2-carboxylic acid conjugate may be diluted with non-derivatized polyoxyethylene alkyl ether to control the density of anthraquinone functional moieties on a CNT surface as illustrated in FIG. 3.

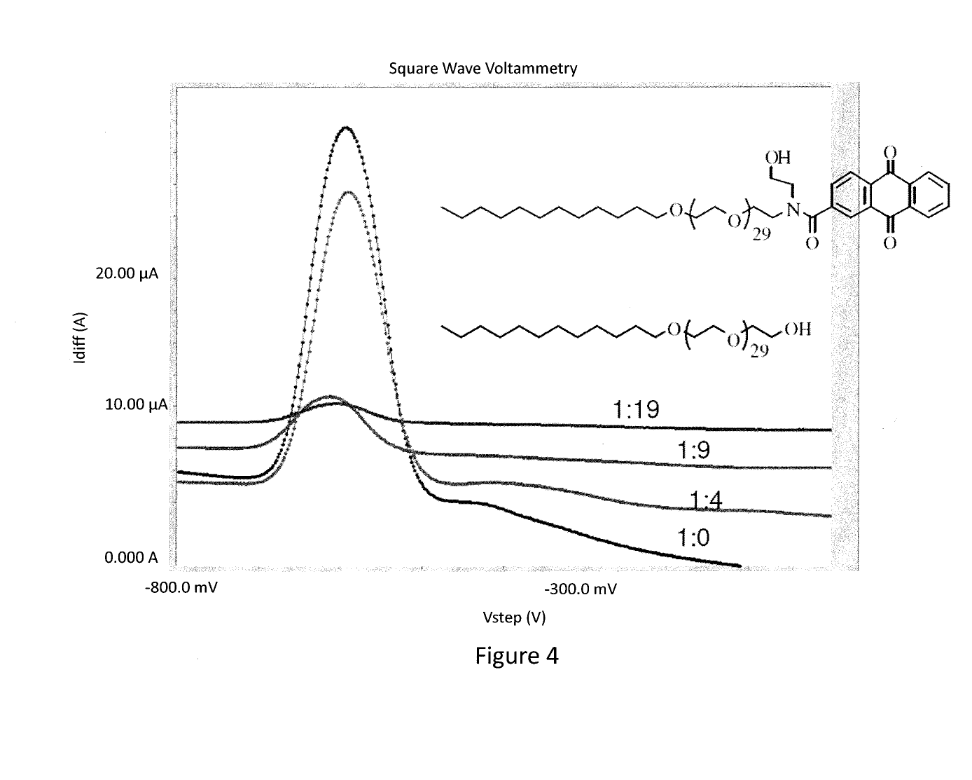

FIG. 4 is a graphical depiction of a square wave voltammogram overlay of CNT nanostructures functionalized with different ratios of polyoxyethylene alkyl ether anthraquinone 2-carboxylic acid conjugate and C12EG30 for the formation of a second layer. With reference to FIG. 4, when N—(C12EG29CH₂CH₂)—N-(2-hydroxyethyl)anthraquinone-2-carboxylic acid amide and C12EG30 were mixed in different ratios (1:0, 1:4, 1:9 and 1:19) for the formation of a second layer on a CNT, it was discovered that the redox signal amplitude in square wave voltammetry (SWV) could be modulated. It should be noted that while the mixtures of only two compounds (N—(C12EG29CH₂CH₂)—N-(2-hydroxyethyl)anthraquinone-2-carboxylic acid amide and C12EG30) are demonstrated in FIG. 4, this should not limit the scope of the claims appended herewith as it is envisioned that mixtures of three or more compounds, including but not limited to the aforementioned compounds, may also be employed to introduce three or more functional groups or functional moieties with controlled density of each functional group or functional moiety.

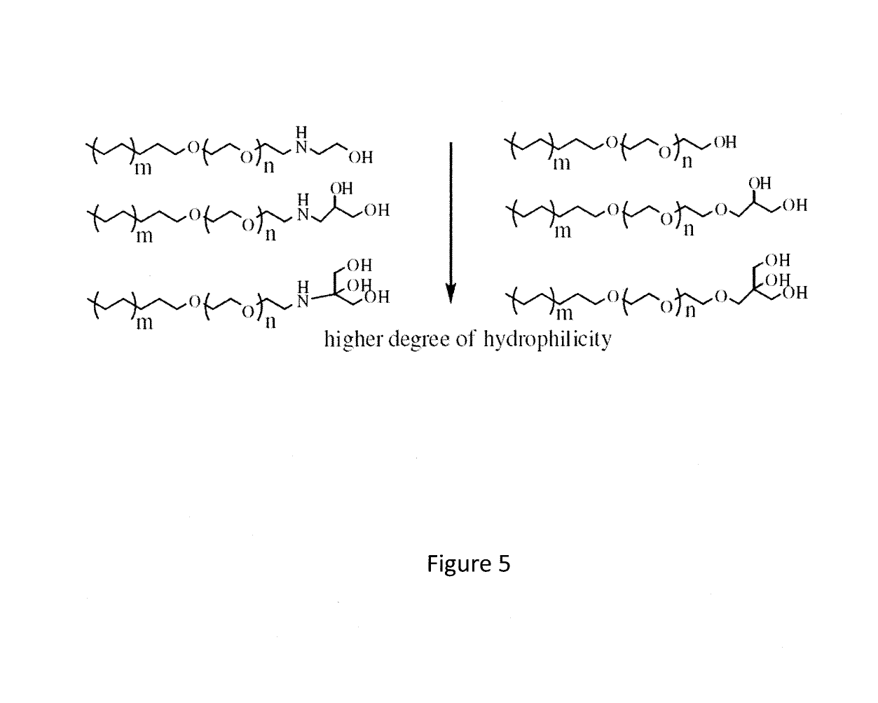

Surface hydrophilicity of exemplary functionalized CNT nanostructures is important for such nanostructures to be used as electrodes since many electrochemical reactions in aqueous solutions require the participation of H⁺ or OH⁻. It follows that one may then control the degree of surface hydrophilicity at the molecular level. Thus, by increasing the number of terminal —OH groups in the polyoxyethylene alkyl ether chain, the degree of hydrophilicity of the subsequently functionalized CNT surface may be increased. For example, the tosylate of polyoxyethylene alkyl ether may be treated with ethanolamine, 2-amino-1,3-propandiol, 3-amino-1,2-propandiol and tris(hydroxymethyl)aminomethane to introduce 1, 2 and 3 terminal —OH groups onto the polyoxyethylene alkyl ether chain. FIG. 5 is a schematic illustration of controlling the number of —OH groups in a bipolar molecule used for the formation of a second layer on a functionalized CNT surface. Of course, many other aminopolyols may be used in embodiments of the present subject matter including, but not limited to, amino saccharides that can be covalently linked to polyoxyethylene alkyl ether chain in similar fashion and such an example should not limit the scope of the claims appended herewith. When used to form the second layer on a CNT surface, these derivatized polyoxyethylene alkyl ethers may lead to a surface having various degrees of hydrophilicity due to the presence of different numbers of terminal —OH groups. Further, the use of primary aminoalcohols may also provide for subsequent derivatization of a resulting secondary amino group with various carboxylic acids including anthraquinone 2-carboxylic acid, 3-(2,5-dimethoxyphenyl)propionic acid and (±)-α-lipoic acid via amide bond formation. It should be noted that other functional groups and functional moieties such as, but not limited to, redox mediator molecules, crown ethers, catalysts, boric acids, carbohydrates, oligonucleotides (DNAs and RNAs), DNA or RNA aptamers, peptide aptamers, proteins such as enzymes and antibodies, quantum dots and nanoparticles, cells, cell organelles, and other cellular components, may be covalently attached to the hydrophilic surface via ester or amide bond formation.

With continued reference to FIG. 5, when surface hydrophilicity is of concern, the tosylate of polyoxyethylene alkyl ether may react with various alcohols in the presence of NaH to afford polyoxyethylene alkyl ether with 0, 1, 2, 3, 4 or more —OH groups per polyoxyethylene alkyl ether chain. Exemplary, non-limiting alcohols may be any monoalkyl alcohol, ethylene glycol, glycerol, erythritol, threitol, pentaerythritol, inositol, xylitol, mannitol and other sugar alcohols. Certain embodiments may also glycosylate the terminal —OH group in polyoxyethylene alkyl ether to covalently link various sugar alcohols or polyols to the polyoxyethylene alkyl ether chain. When such exemplary bipolar molecules are used in the second layer of a CNT nanostructure according to embodiments of the present subject matter, the density of —OH groups may be controlled and the surface hydrophilicity modulated as desired.

One embodiment may modulate and/or control the density of various surface functional groups and functional moieties by mixing a bipolar compound containing the functional groups and/or functional moieties described herein with a similar bipolar compound containing no such functional groups and/or functional moieties according to a specific ratio (e.g., 1:1, 1:2, etc.) in a solution used for the second layer functionalization of an exemplary CNT surface. Further, more than two compounds may also be utilized to simultaneously introduce functional groups with desired density.

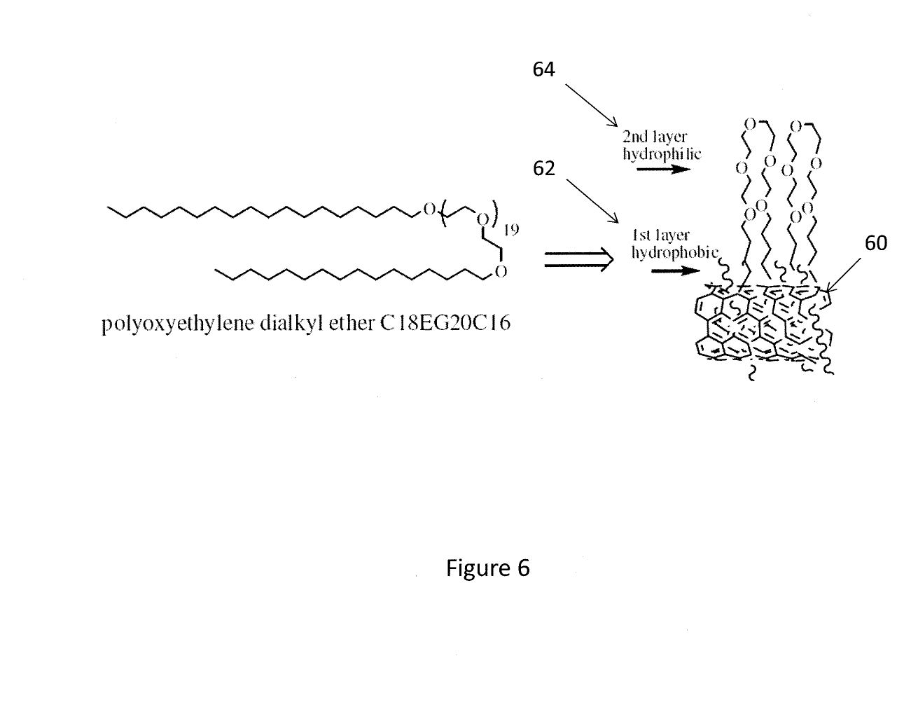

Due to the size and/or polarity of a respective functional group, it may be difficult to construct a second layer structure having certain functional groups attached to the polyoxyethylene alkyl ether chain and/or it may be difficult to ensure that certain functional groups are exposed on the outer surface of the second layer. For example, when the functional moiety of a prospective functional group is an enzyme molecule, it may be difficult to eliminate a hydrophobic interaction between the alkyl chain in the polyoxyethylene alkyl ether and the enzyme molecule if the enzyme molecule is first covalently attached to the polyoxyethylene alkyl ether simply due to the sheer size of the enzyme molecule. Further, if the polarity of a certain functional group is similar to the alkyl chain in a polyoxyethylene alkyl ether, when forming the second layer above the protective first layer on an exemplary CNT surface the hydrophobic functional group may not necessarily separate from the alkyl chain and as a result may be buried underneath the hydrophilic polyoxyethylene structure. FIG. 6 is a schematic illustration of depositing a polyoxyethylene dialkyl ether on a CNT surface to form a second layer on a functionalized CNT surface. With reference to FIG. 6, the surface of an exemplary functionalized CNT nanostructure electrode 60, having a protective first layer 62 and a polyoxyethylene dialkyl ether C18EG20C16 in the second layer 64, may be hydrophilic thereby providing an indication that the polyoxyethylene portion thereof is exposed on the outermost surface. In other instances, when the prospective functional group is relatively unstable under the conditions for the formation of the second layer, it may be desirable that the functional group be covalently attached after the second layer structure is established on the CNT surface.

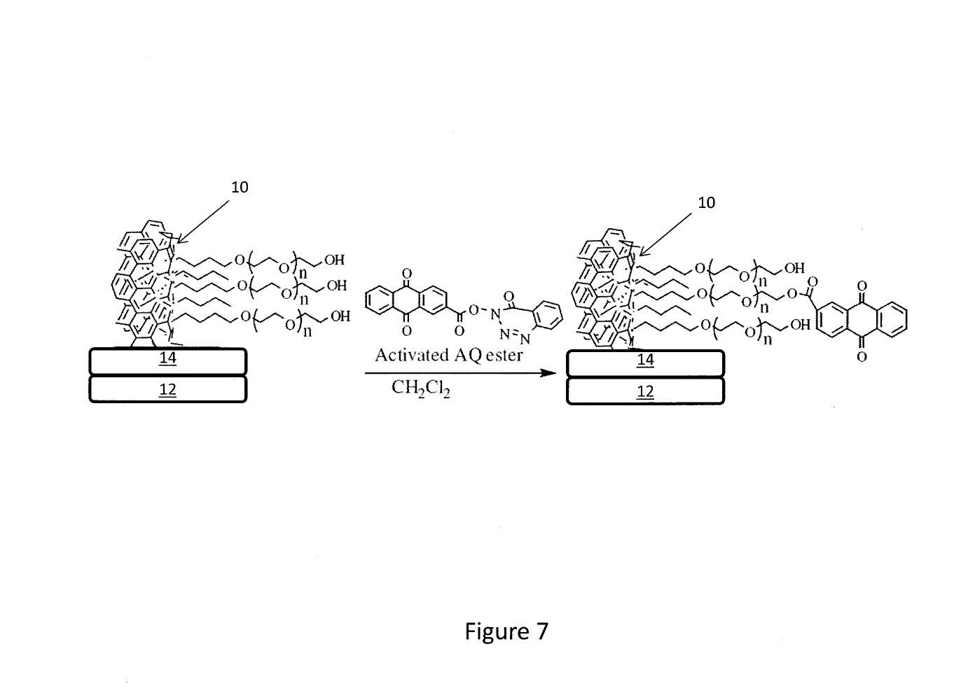

FIG. 7 is an illustration of an exemplary structure of a hydrophilic CNT nanostructure surface and a covalent functionalization of surface —OH groups with an activated anthraquinone ester. With reference to FIG. 7, an exemplary method may establish a hydrophilic platform on a CNT nanostructure 10 amenable for subsequent covalent attachment of various functional groups regardless of their size, polarity, hydrophobicity/hydrophilicity, and/or stability under elevated temperatures. In one embodiment, an exemplary CNT nanostructure electrode 10 may be protected with n-octadecane to form the first protective layer, followed by the deposition of molecules such as C12EG30, (2,2,2-trimethylol) ethylamine tri(3,6,9,12-tetraoxaicosanyl)ether or dioctadecylamine [(n-C₁₈H₃₈)₂NH] to form a second layer with —OH groups (see FIG. 7) or —NH₂ or —NH— groups. Of course, such exemplary groups may be useful for covalent attachment of other functional groups or functional moieties. For example, various carboxylic acids may be introduced to the surface via ester and amide bond formation, and other functional groups and functional moieties including, but not limited to, redox mediator molecules, crown ethers, catalysts, boric acids, carbohydrates, oligonucleotides (DNAs and RNAs), DNA or RNA aptamers, peptide aptamers, peptides and proteins such as enzymes and antibodies, quantum dots and nanoparticles, cells, cell organelles, and other cellular components, may also be covalently linked to the amino and/or hydroxyl groups on the hydrophilic surface.

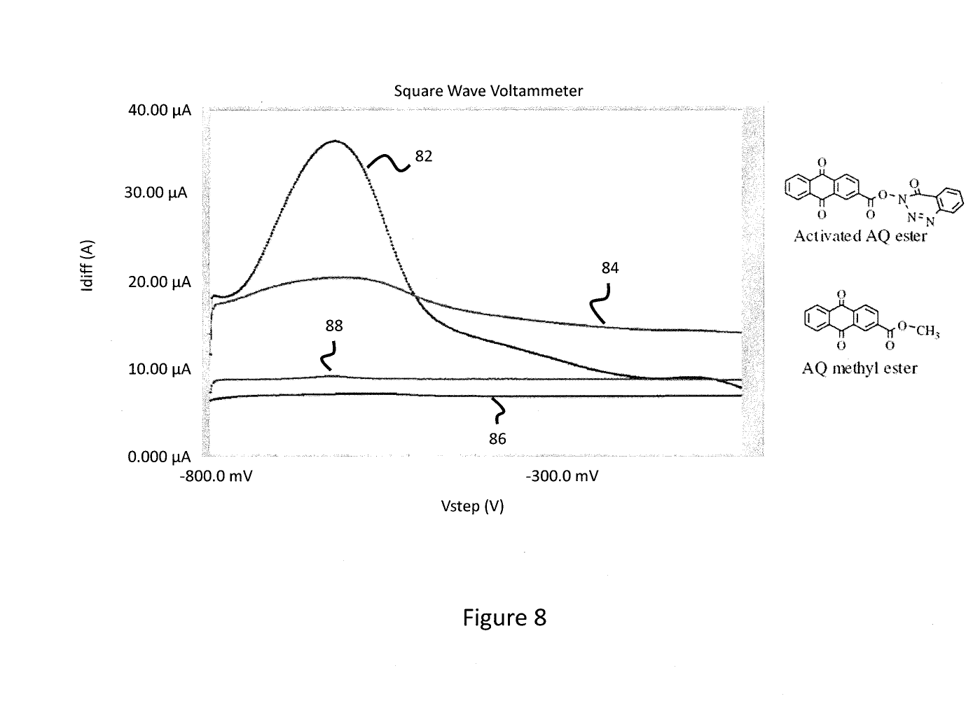

FIG. 8 is a graphical depiction of a square wave voltammogram overlay of differently functionalized CNT nanostructure electrodes. With reference to FIG. 8 and continued reference to FIG. 7 to illustrate certain principles underlying embodiments of the present subject matter, a series of CNT nanostructures 10 were used to demonstrate the covalent nature of an exemplary functionalization process with functional groups such as —OH and —NH₂ in the second layer. These CNT nanostructures 10 were first protected with n-octadecane in the first layer 62 and then deposited with C12EG30 to form the second layer 64 with —OH groups on the surface thereof. When the surface was treated with an activated AQ ester, the CNT nanostructure electrode 10 yielded a strong redox signal for the AQ as graphically illustrated by a first trace 82. When a similar surface was treated with a solution with anthraquinone 2-carboxylic acid methyl ester (AQ methyl ester) and a trace of the activated AQ ester, the CNT nanostructure electrode 10 subsequently yielded a considerably smaller redox signal for the AQ as graphically illustrated by a second trace 84. When a CNT nanostructure 10 was only protected with n-octadecane and no deposition of C12EG30 occurred for the second layer (hence no —OH groups on the nanostructure surface), subsequent treatment of the electrode with activated AQ ester under identical condition resulted in no redox signal for the AQ as illustrated by a third trace 86. Finally, another CNT nanostructure 10 having n-octadecane and then C12EG30 deposition was treated with only anthraquinone 2-carboxylic acid methyl ester, and the resulting CNT nanostructure electrode 10 subsequently did not produce a redox signal for the AQ as graphically illustrated by a fourth trace 88 thereby indicating no non-specific adsorption of anthraquinone 2-carboxylic acid methyl ester onto the hydrophilic surface. These experiments indicate the covalent nature of the hydrophilic surface functionalization of certain embodiments of the present subject matter and provide evidence that exemplary CNT nanostructures according to embodiments of the present subject matter having poly(ethylene glycol) functionality on the surface thereof following a layer-by-layer process may be resistant to non-specific adsorption.

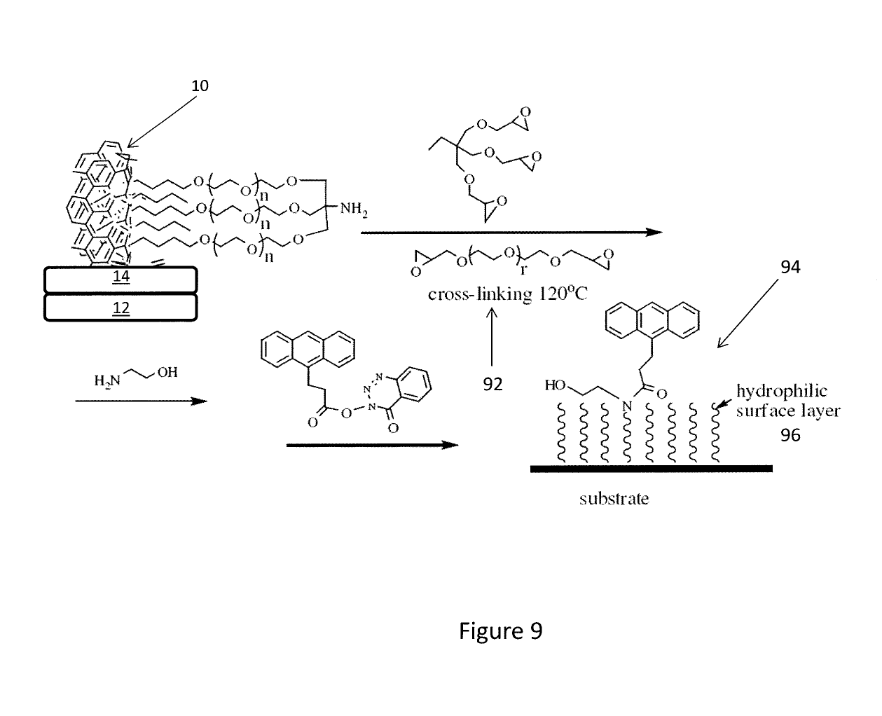

FIG. 9 is a schematic illustration of an exemplary layer-by-layer introduction of various functional groups onto a CNT nanostructure surface. With reference to FIG. 9, ring opening reactions of epoxide may be advantageously employed using a CNT surface having a second layer with —OH groups or —NH₂ or —NH— groups that readily react with polyetheneglycol diglycidyl ether and trimethylolpropane triglycidyl ether. Other glycidyl ethers can also be used including trimethylolethane triglycigyl ether, glycerol diglycidyl ether, glycerol triglycidyl ether, glycerol polyglycidyl ether, diglycerol polyglycidyl ether, polyglycerol polyglycidyl ether, pentaerythritol glycidyl ether, pentaerythritol polyglycidyl ether, sorbitol polyglycidyl ether and so on. Exemplary crosslinking 92 may result in a new polymeric layer (e.g., third layer) 94 with excess epoxide groups. When treated with amino alcohols such as ethanolamine, 2-amino-1,3-propandiol, 3-amino-1,2-propandiol and tris(hydroxymethyl)aminomethane, secondary amine —NH— and 1, 2 or 3 terminal —OH groups may be introduced onto the CNT surface. Of course, many other aminopolyols including, but not limited to, aminosaccharides may be employed in similar fashion and such a disclosure should not limit the scope of the claims appended herewith. The resulting amino and/or hydroxyl groups may also be derivatized with various carboxylic acids including 3-(anthracen-9-yl)propionic acid, anthraquinone 2-carboxylic acid, 3-(2,5-dimethoxyphenyl)propionic acid and (±)-α-lipoic acid. Similarly, other functional groups and functional moieties including, but not limited to, redox mediator molecules, crown ethers, catalysts, boric acids, carbohydrates, oligonucleotides (DNAs and RNAs), DNA or RNA aptamers, peptide aptamers, peptides and proteins such as enzymes and antibodies, quantum dots and nanoparticles, cells, cell organelles, and other cellular components, may also be covalently linked to the amino and/or hydroxyl groups on the hydrophilic surface layer 96.

In embodiments of the present subject matter where it may be necessary to further derivatize functional groups or functional moieties in the second layer on a CNT surface, the CNT nanostructure may be treated with reagents in an appropriate solvent, e.g., activated AQ ester in CH₂Cl₂ for a predetermined period as described in examples above. Upon completion of this further reaction, the CNT nanostructure on the respective substrate may be rinsed with a solvent (e.g., THF), dried in air, and then wire-bonded and assembled for testing. In an alternative embodiment, a CNT nanostructure functionalized with the first layer and second layer may be treated with reagents to form a third layer (e.g., via cross-linking) followed by subsequent transformations to incorporate additional functional groups or functional moieties. For example, after the formation of a C12EG30 layer on top of a first n-octadecane layer, a CNT nanostructure on a substrate may be treated with a mixture solution of polyethylene glycol diglycidyl ether (PEGDGE) and trimethylolpropane triglycidyl ether (TMPTGE) in THF (25 mM/25 mM, 2×5 μL), dried in air, and then warmed at 120° C. in a tightly capped vial under Argon for approximately five hours. The CNT nanostructure on the substrate may then be cooled to room temperature, rinsed with THF to remove excess PEGDGE and TMPTGE on the substrate. The CNT nanostructure may then be dried in air and placed in a tightly capped vial with a mixture of Tris (121 mg) in DMF (1 mL) under Argon atmosphere and warmed at 80° C. for approximately 24 hours before removal from the DMF solution. This nanostructure may then be rinsed with MeOH and THF and dried in air. In a separate vial, anthraquinone 2-carboxylic acid (9 mg, 0.0356 mmol) may be mixed with diisopropylethylamine (10.4 μL, 0.071 mmol) and 3-diethoxyphosphoryloxy-1,2,3-benzotriazin-4(3H)-one (10.6 mg, 0.0356 mmol) resulting in a yellow solution. The CNT nanostructure may then be placed in the yellow solution for approximately 16 hours and rinsed with THF and dried. An exemplary CNT nanostructure functionalized in this manner may then, for example, be used as voltammetric pH sensor with long-term stability. Of course, such a process is exemplary only and should not limit the scope of the claims appended herewith.

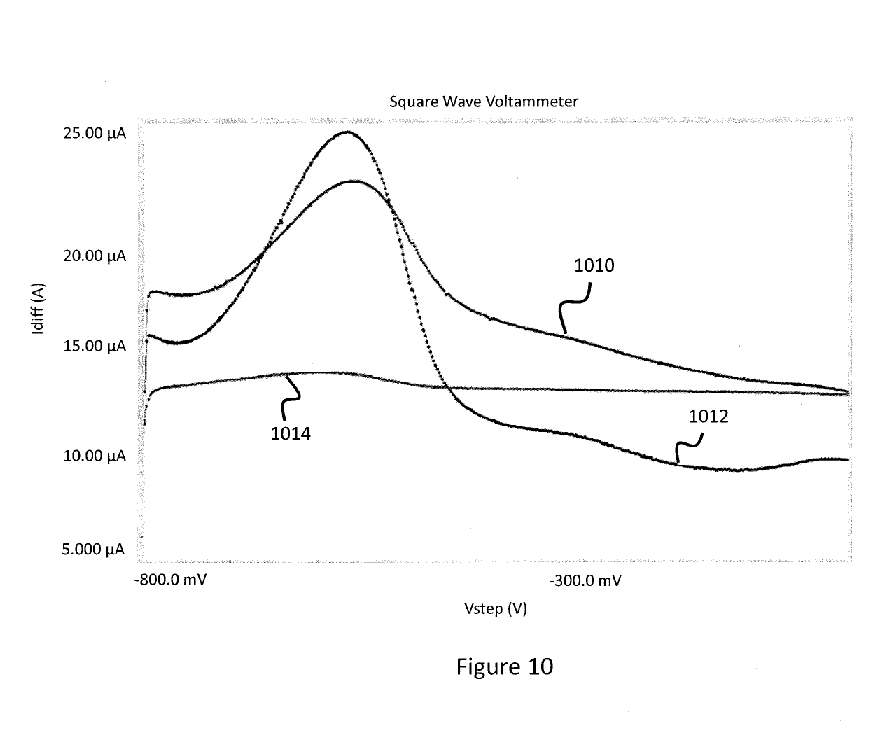

FIG. 10 is a graphical depiction of a square wave voltammogram overlay for various embodiments of the present subject matter. With reference to FIG. 10 and continued reference to FIG. 9, in another embodiment a series of bipolar molecules having —OH, —NH₂ and secondary amine groups may be used for the deposition of the second layer on an exemplary CNT surface. For example, (2,2,2-trimethylol) ethylamine tri(3,6,9,12-tetraoxaeicosanyl)ether was employed for this purpose as schematically illustrated by the generic process depicted in FIG. 9. Dialkyl amine including dioctadecylamine may also be applied to achieve a similar level of layer-by-layer surface functionalization. Polyoxyethylene alkyl ethers such as, but not limited to, C12EG30 and C12EG30NHCH₂CH₂OH may also be used as the second layer material for cross-linking to construct an exemplary third layer on a CNT surface. Upon subsequent epoxide ring opening and covalent bond formation with an activated AQ ester, a strong redox signal may be observed by a first and second trace 1010, 1012 in FIG. 10. When no cross-linking step was employed as illustrated by a third trace 1014, subsequent treatment with tris(hydroxymethyl)aminomethane (Tris) in dimethylformamide (DMF) at an elevated temperature (80° C. for 16 h) resulted in the removal of uncrosslinked C12EG30 and thus little or no —OH groups remained on the CNT surface for AQ functional moiety attachment which appears to explain the small redox signal for the AQ observed in the third trace 1014. These results provide an indication of the formation of a third layer by cross-linking on the CNT surface. Additionally, a cross-linked third layer may also stabilize the anchoring of functional groups and functional moieties on an exemplary CNT surface and may maintain the structural integrity of the surface layers thereby rendering the surface less prone to non-specific adsorption. Exemplary CNT surfaces functionalized in this fashion may provide excellent long-term stability and are suitable for subsequent introduction of proteins and enzymes. Of course, other cross-linking and polymerization approaches may be employed by embodiments of the present subject matter for the construction of a third layer and such an example should not limit the scope of the claims appended herewith. For example, it is envisioned that polymer brushes may be grown on top of the second or third layer of an exemplary CNT surface following the layer-by-layer approach and, with the appropriate chemistries, construction of a multilayer, organized structure possessing a controlled layer thickness may be performed.

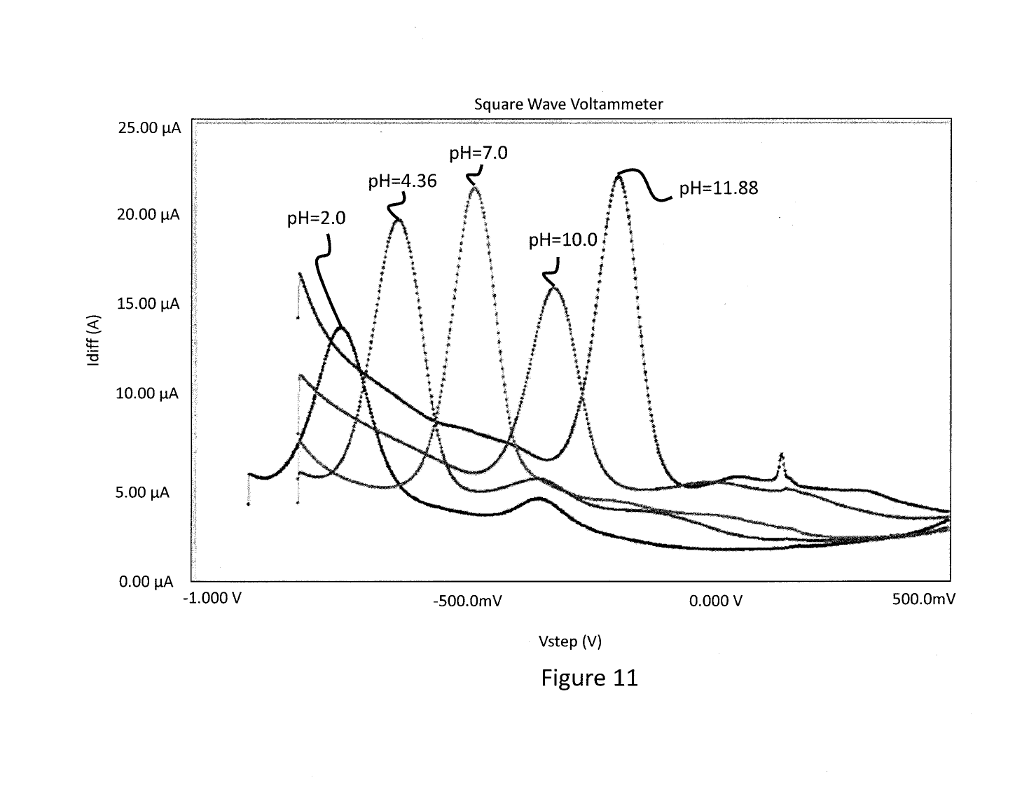

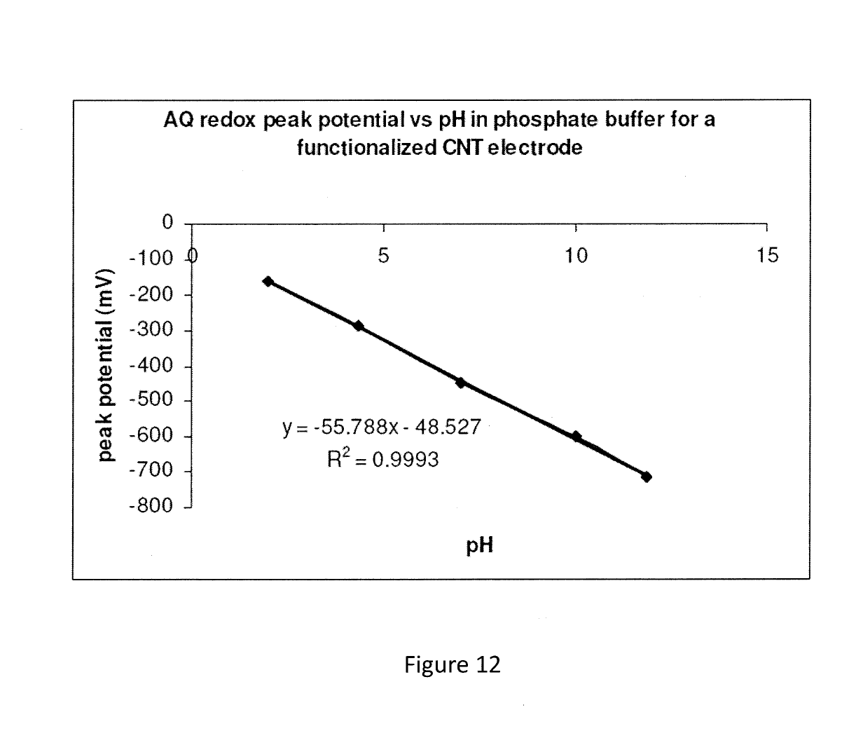

FIG. 11 is a graphical depiction of a square wave voltammogram overlay of a CNT nanostructure electrode functionalized with anthraquinone in buffer solutions at various pHs. FIG. 12 is a plot of an anthraquinone square wave voltammogram redox peak potential versus buffer solution pH for a CNT nanostructure electrode functionalized via an embodiment of the present subject matter. With reference to FIGS. 11 and 12, exemplary functionalized carbon nanostructures may be used as sensing elements in various applications. For example, a voltammetric pH sensor was fabricated using a CNT nanostructure on a silicon substrate functionalized using an exemplary layer-by-layer approach with anthraquinone 2-carboxylic acid. When subjected to square-wave voltammetry (SWV) in a 0.05 M phosphate buffer solution with 0.1 M NaClO₄ as a supporting electrolyte at various pHs (2.0, 4.36, 7.0, 10.0 and 11.88), the sensor electrode generated symmetrical redox peaks for the respective pHs. In this embodiment of a surface functionalized CNT nanostructure as a voltammetric pH sensor, SWV measurements were recorded using a Reference 600 potentiostat 5.61 with a standard three-electrode configuration, consisting of a Ag/AgCl reference electrode, a carbon rod auxiliary counter electrode, and a CNT nanostructure on Si substrate as working electrode in a specially designed electrochemical cell. The exemplary CNT nanostructure was functionalized with redox mediator molecules using an exemplary layer-by-layer approach, and then exposed to different pH solutions (about 5 mL) in an electrochemical cell. Different pH solutions were then prepared in deionized water as follows: pH 2.0, 0.05 M H₃PO₄ adjusted with 10% NaOH solution; pH 4.36, 0.05 M NaH₂PO₄; pH 7.0, 0.05 M Na₂HPO₄ adjusted with 0.05 M NaH₂PO₄; pH 10.0, 0.05 M Na₂HPO₄ adjusted with 10% NaOH solution; pH 11.88, 0.05 M Na₂HPO₄ adjusted with 10% NaOH solution. NaClO₄ may be added into these solutions as a supporting electrolyte to a concentration of 0.1 M. The pH values of these solutions may be obtained using a pH meter. SWV were performed with the following parameters: frequency 10 Hz, step potential 2 mV, amplitude 25 mV within the potential range of −1.0 V and 0.5 V. Square wave (SW) voltammograms recorded using an AQ functionalized CNT nanostructure electrode were overlaid and are illustrated in FIG. 11. These voltammograms indicate that as pH increases from pH 2 to pH 11.88, the AQ redox peak shifts to more negative potential.

A plot of redox peak potential against pH illustrated in FIG. 12 provides a linear, Nernstian response having a slope of −55.8 mV/pH and linearity R² of 0.9993, substantially close to the theoretical slope of −59.1 mV/pH. FIG. 12 thus reflects the plot of the AQ redox peak potential versus solution pH demonstrating a linear response from pH 2 to pH 11.88 with a slope of −55.788 mV per pH unit. It is apparent that an exemplary CNT nanostructure electrode functionalized using a layer-by-layer approach with redox mediator molecules such as, but not limited to, AQ may be advantageously utilized as pH sensors for aqueous solutions.

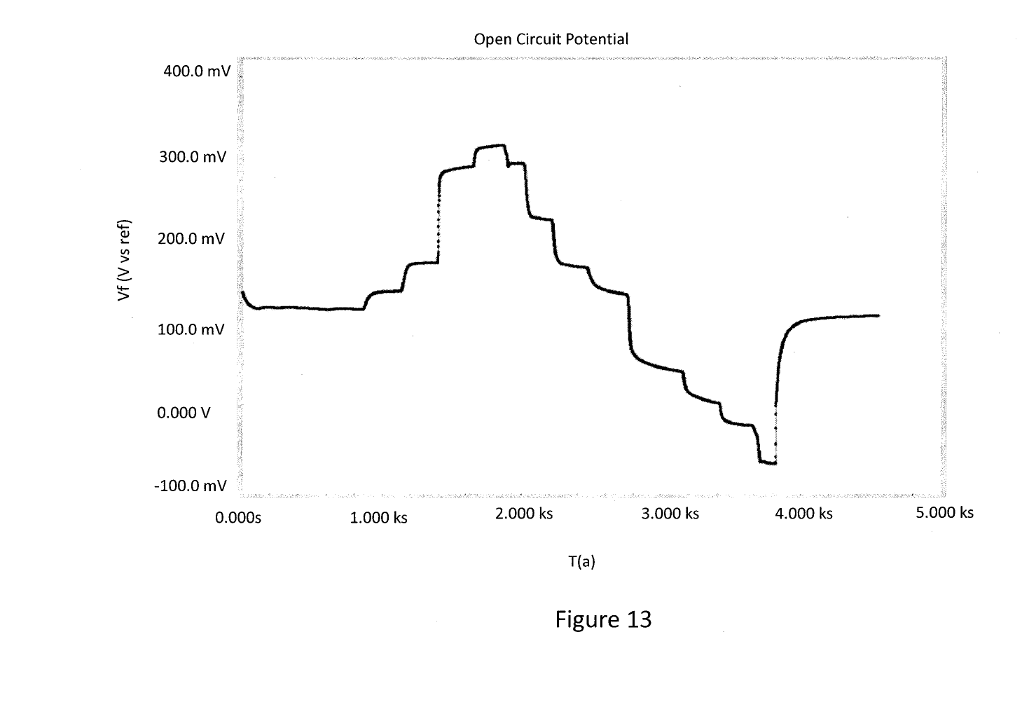

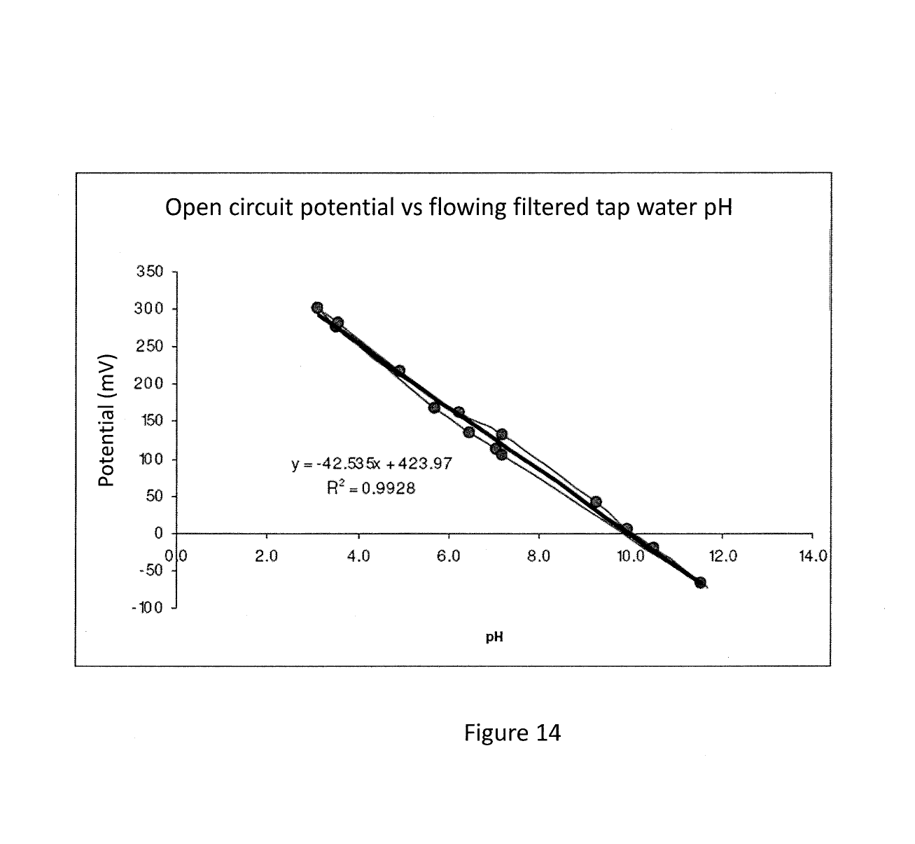

FIG. 13 is a graphical depiction of an open circuit potential of a CNT nanostructure electrode functionalized using an embodiment of the present subject matter. FIG. 14 is a plot of open circuit potential versus pH for flowing tap water using an embodiment of the present subject matter. With reference to FIGS. 13 and 14, a potentiometric pH sensor was fabricated using a CNT nanostructure on a silicon substrate functionalized using an exemplary layer-by-layer approach with n-octadecane for the first layer and then polyoxyethylene alkyl ether Brij®35 (C12EG30) for the second layer. The structure was then placed in a polydimethylsiloxane (PDMS) flow cell and used as a potentiometric pH sensor for flowing tap water with low conductivity. As shown in FIG. 13, open circuit potential (OCP) was measured against an Ag/AgCl reference electrode (RE-6, BASi Analytical Instruments) placed 3 mm above the CNT nanostructure electrode. To generate the results depicted in FIG. 13, OCP measurements were recorded using a Reference 600 potentiostat 5.61 with a two-electrode configuration having an Ag/AgCl reference electrode and a CNT nanostructure on a silicon substrate as working electrode in a specially designed PDMS flow cell. The CNT nanostructure may be functionalized using an exemplary layer-by-layer approach and then exposed to flowing tap water (after filtration over activated carbon to remove free chlorine in tap water) at different pH. The pH of the flowing tap water may be monitored using a glass pH meter and adjusted in a reservoir with dilute HCl or NaOH solutions upon constant stirring. Thus, FIG. 13 illustrates the OCP change with different water pH whereby at a given pH, the CNT nanostructure electrode possessed a definite potential. It is shown that the potential shifted to more negative as the solution pH increased.

As illustrated in FIG. 14, it was discovered that the OCP of the CNT electrode provided a linear response to pH in flowing tap water. This plot of the OCP against flowing tap water pH produced a linear response (R²=0.9928) with a gradient of −45.2 mV per pH unit between pH 3 and 12. Thus, the use of an exemplary CNT nanostructure electrode as a potentiometric pH sensor is evident, and such an exemplary potentiometric pH sensor may find utility in water or other aqueous solutions with low ionic concentrations as well as under pressure.

While embodiments have been heretofore described in connection with amperometric pH sensors, potentiometric pH sensors, voltammetric pH sensors, electrodes and other sensors, the scope of the claims appended herewith should not be so limited. For example, it is envisioned that embodiments of the present subject matter may find utility in the biometric industry and as intracorporeal electrodes and sensors.

Additionally, data, commands and other information or messages may be sent or received, wirelessly or via wire-line depending upon the application, from or to various electrodes and/or sensors utilizing an exemplary system. For example, an exemplary monitoring system may collect information from a sensor monitoring the pH of a remote or local fluid system and may provide such information to a user or to a database for real-time or stored use. Further, an exemplary monitoring system may collect information transmitted wirelessly from an intracorporeal sensor or matrix of sensors or electrodes. Such provision (i.e., transmission) of information may be via any known mode of transmission (e.g., wireless or wire-line, as applicable). Such information may also be provided directly to a user or may be provided to a user via a processor for manipulation and/or storage thereof. Of course, the processor and supporting systems may also be employed to provide messages and/or commands to the remote or local sensor or electrode as the need arises. Thus, it is envisioned that embodiments may be implemented using a general purpose computer programmed in accordance with the principals discussed herein. It is also envisioned that embodiments of the subject matter and the functional operations described in this specification may be implemented in or utilize digital electronic circuitry, or in computer software, firmware, or hardware, including the structures disclosed in this specification and their structural equivalents, or in combinations of one or more of them. Thus, embodiments of the subject matter described in this specification can be implemented in or utilize one or more computer program products, i.e., one or more modules of computer program instructions encoded on a tangible program carrier for execution by, or to control the operation of, data processing apparatus. The tangible program carrier can be a computer readable medium. The computer readable medium can be a machine-readable storage device, a machine-readable storage substrate, a memory device, or a combination of one or more of them.

To note, the term “processor” encompasses all apparatus, devices, and machines for processing data, including by way of example a programmable processor, a computer, or multiple processors or computers. The processor can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Of course, the general processes described by monitoring systems herein may be performed by one or more programmable processors executing one or more computer programs to perform functions by operating on input data and generating output. These processes may also be performed by special purpose logic circuitry, e.g., a field programmable gate array (FPGA) or an application specific integrated circuit (ASIC). Processors suitable for the execution of an exemplary computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more data memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, to name just a few.

Computer readable media suitable for storing computer program instructions and data include all forms of data memory including non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, exemplary systems according to embodiments of the subject matter may be implemented on a computer having a display device, e.g., a cathode ray tube (CRT) or liquid crystal display (LCD) monitor, for displaying information to the user and a keyboard and a pointing device, e.g., a mouse or a trackball, by which the user can provide input to the computer. Other kinds of devices can be used to provide for interaction with a user as well; for example, input from the user can be received in any form, including acoustic, speech, or tactile input.

Embodiments of the subject matter described in this specification may also be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system may be interconnected by any form or medium of digital data communication, e.g., a communication network. Examples of communication networks include a local area network (LAN) and a wide area network (WAN), e.g., the Internet. The computing system may also include clients and servers as the need arises. A client and server are generally remote from each other and typically interact through a communication network. The relationship of client and server arises by virtue of computer programs running on the respective computers and having a client-server relationship to each other.

It may be emphasized that the above-described embodiments, particularly any “preferred” embodiments, are merely possible examples of implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

While this specification contains many specifics, these should not be construed as limitations on the scope of the claimed subject matter, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

As shown by the various configurations and embodiments illustrated in FIGS. 1-14, devices and methods for layer-by-layer carbon nanostructure surface functionalization have been described.

While preferred embodiments of the present subject matter have been described, it is to be understood that the embodiments described are illustrative only and that the scope of the invention is to be defined solely by the appended claims when accorded a full range of equivalence, many variations and modifications naturally occurring to those of skill in the art from a perusal hereof.

What is claimed is:

1. A method of functionalizing carbon nanostructures comprising the steps of:
providing a carbon nanostructure having a first protective layer on a surface of the structure; and
forming a functional second layer over the first protective layer,
wherein the second layer comprises a bipolar molecule with functional groups or functional moieties.

2. The method of claim 1 wherein the step of providing a carbon nanostructure further comprises contacting the carbon nanostructures with a composition comprising an alkyl protective moiety under conditions that permit the formation of an alkyl protective layer.

3. The method of claim 2 wherein the alkyl protective moiety is selected from the group consisting of linear alkanes, branched alkanes, alkenes, alkenes containing 10 to 50 carbon atoms, alkenes substituted with one or more halogen atoms, n-octadecane, n-dodecane, eicosane and hexatriacontane, and combinations thereof.

4. The method of claim 1 wherein the bipolar molecule comprises a compound having the general formula: 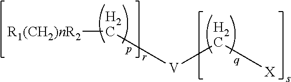
wherein
R₁ represents hydrogen or a C₁₋₅₀ straight or branched alkyl or alkenyl, which is optionally substituted with one or more halogen atoms;
R₂ represents a single bond, an aromatic or alicyclic group, —(OCH₂CH₂)ₘ—, —(OCH₂CH₂CH₂)ₘ—, or —[OCH₂CH(CH₃)]ₘ—, where m and n are each independently 0 to 500;
X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, —NH₂, —NR′R″, —N(COR′)R″, —N+R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;
R represents R₁, R₁(CH₂)ₙR₂ or —(CH₂)ₙR₂X;
R′, R″, R′″ are each hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]ₙR;
p, q are each an integral number between 0 and 10;
r, s are each an integral number between 1 and 4, and 1<r+s<=4; and
V represents a single bond, C, CH, CH₂, Si, N, NH, P, (P═O) or O.

5. The method of claim 1 wherein the bipolar molecule comprises a compound with two linked subunits having the general formula: 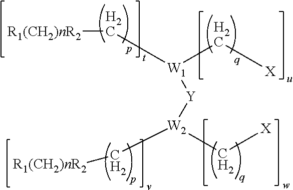
wherein
R₁ represents hydrogen or a C₁₋₅₀ straight or branched alkyl or alkenyl, which is optionally substituted with one or more halogen atoms;
R₂ represents a single bond, an aromatic or alicyclic group, —(OCH₂CH₂)ₘ—, —(OCH₂CH₂CH₂)ₘ—, or —[OCH₂CH(CH₃)]ₘ—, where m and n are each independently 0 to 500;
X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, —NH₂, —NR′R″, —N(COR′)R″, —N⁺R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;
R represents R₁, R₁(CH₂)ₙR₂ or —(CH₂)ₙR₂X;
R′, R″, R′″ are each hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]ₙR;
p, q are each an integral number between 0 and 10;
t, v are each an integral number between 1 and 3, u and w are each an integral number between 0 and 2, and 1<=t+u<=3, and 1<=v+w<=3;
W₁, W₂ each represents C, CH, CH₂, Si, N, NH, P, (P═O) or O; and
Y represents a single bond or a divalent linker that comprises: C₁₋₅₀ alkyl, alkenyl or aromatic group which is optionally substituted with one or more X; —(OCH₂CH₂)ₘ—, —(OCH₂CH₂CH₂)ₘ—, or —[OCH₂CH(CH₃)]ₘ—, where m and n may each be independently 0 to 500.

6. The method of claim 5 wherein the bipolar molecule further comprises a compound with more than two subunits connected in linear or cyclical fashion with multiple linker groups.

7. The method of claim 1 wherein the functional groups or functional moieties are selected from the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof.

8. The method of claim 1 further comprising the step of derivatizing the functional groups or functional moieties in the second layer to form covalent bonds with other functional groups or moieties.

9. The method of claim 8 wherein the other functional groups or moieties are selected the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof.

10. The method of claim 1 wherein the step of forming a functional second layer further comprises controlling the density of the functional groups or functional moieties by applying bipolar molecules having a predetermined ratio of functional groups or functional moieties.

11. The method of claim 1 further comprising the step of modulating the surface hydrophilicity of the carbon nanostructure using polyoxyethylene alkyl ethers having one or more hydroxyl groups.

12. The method of claim 1 further comprising the step of cross-linking the functional groups or functional moieties in the second layer to form a third layer on the surface of the carbon nanostructure.

13. The method of claim 12 further comprising the step of introducing additional functional groups or functional moieties onto the carbon nanostructure using the third layer.

14. The method of claim 1 wherein the functionalized carbon nanostructure is selected from the group consisting of a sensing element, a voltammetric pH sensor, a potentiometric pH sensor, an electrode, an amperometric pH sensor, a biometric sensor, a biometric electrode, an intracorporeal sensor, and an intracorporeal electrode.

15. A carbon nanostructure comprising:
(a) a substrate having one or more carbon nanotubes situated on a surface of said substrate;
(b) a first protective layer covering portions of said carbon nanotubes and said substrate; and
(c) a functional second layer over said first protective layer,
wherein the second layer comprises a bipolar molecule with functional groups or functional moieties.

16. The nanostructure of claim 15 wherein the carbon nanostructure is selected from the group consisting of a sensing element, a voltammetric pH sensor, a potentiometric pH sensor, an electrode, an amperometric pH sensor, a biometric sensor, a biometric electrode, an intracorporeal sensor, and an intracorporeal electrode.

17. The nanostructure of claim 15 wherein the first protective layer comprises an alkyl protective moiety.

18. The nanostructure of claim 17 wherein the alkyl protective moiety is selected from the group consisting of linear alkanes, branched alkanes, alkenes, alkenes containing 10 to 50 carbon atoms, alkenes substituted with one or more halogen atoms, n-octadecane, n-dodecane, eicosane and hexatriacontane, and combinations thereof.

19. The nanostructure of claim 15 wherein the bipolar molecule comprises a compound having the general formula: 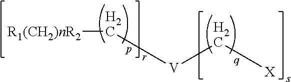
wherein
R₁ represents hydrogen or a C₁₋₅₀ straight or branched alkyl or alkenyl, which is optionally substituted with one or more halogen atoms;
R₂ represents a single bond, an aromatic or alicyclic group, —(OCH₂CH₂)ₘ—, —(OCH₂CH₂CH₂)ₘ—, or —[OCH₂CH(CH₃)]ₘ, where m and n are each independently 0 to 500;
X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, —NH₂, —NR′R″, —N(COR′)R″, —N+R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;
R represents R₁, R₁(CH₂)ₙR₂ or —(CH₂)ₙR₂X;
R′, R″, R′″ are each hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]R;
p, q are each independently an integral number between 0 and 10;
r, s are each an integral number between 1 and 4, and 1<r+s<=4; and
V represents a single bond, C, CH, CH₂, Si, N, NH, P, (P═O) or O.

20. The nanostructure of claim 15 wherein the bipolar molecule comprises a compound with two linked subunits having the general formula: 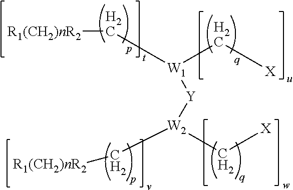
wherein
R₁ represents hydrogen or a C₁₋₅₀ straight or branched alkyl or alkenyl, which is optionally substituted with one or more halogen atoms;
R₂ represents a single bond, an aromatic or alicyclic group, —(OCH₂CH₂)ₘ—, —(OCH₂CH₂CH₂)ₘ—, or —[OCH₂CH(CH₃)]ₘ, where m and n are each independently 0 to 500;
X represents hydrogen, halogen, maleimido group, epoxide, —C≡CH, —N₃, —CN, —OH, —OSO₃⁻, —OR, —SH, —SR, —S—S—R, —SO₃H, —SO₃R, —SO₃⁻, —PO₃H₂, —PO₃H⁻, —(PO₃)²⁻, —P(═O)(—OR′)(OR″), —OPO₃H₂, —OPO₃H⁻, —O(PO₃)²⁻, —CHO, —COR, —COOH, —COO⁻, —COOR, —CONR′R″, —CONHNH₂, —NH₂, —NR′R″, —N(COR′)R″, —N+R′R″R′″, —N⁺C₅H₅, —(OCH₂CH₂)ₘ—OR, —(OCH₂CH₂CH₂)ₘ—OR, —[OCH₂CH(CH₃)]ₘ—OR, a polyol, a monosaccharide, a disaccharide or a polyethylene oxide derivative thereof;
R represents R₁, R₁(CH₂)ₙR₂ or —(CH₂)ₙR₂X;
R′, R″, R′″ are each hydrogen, alkyl, cycloalkyl, alkyl and cycloalkyl substituted by one or more hydroxyl groups, alkyl and cycloalkyl substituted by one or more carboxylic groups, —(CH₂CH₂O)ₙR, —(CH₂CH₂CH₂O)ₙR, or —[CH₂CH(CH₃)O]ₙR;
p, q are each an integral number between 0 and 10;
t, v are each an integral number between 1 and 3, u and w are each an integral number between 0 and 2, and 1<=t+u<=3, and 1<=v+w<=3;
W₁, W₂ each represent C, CH, CH₂, Si, N, NH, P, (P═O) or O; and
Y represents a single bond or a divalent linker that comprises: C₁₋₅₀ alkyl, alkenyl or aromatic group which is optionally substituted with one or more X; —(OCH₂CH₂)ₘ—, —(OCH₂CH₂CH₂)ₘ—, or —[OCH₂CH(CH₃)]ₘ—, where m and n may each be independently 0 to 500.

21. The nanostructure of claim 20 wherein the bipolar molecule further comprises a compound with more than two subunits connected in linear or cyclical fashion with multiple linker groups.

22. The nanostructure of claim 15 wherein the functional groups or functional moieties are selected from the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof.

23. The nanostructure of claim 15 further comprising a third layer on said surface of the carbon nanostructure.

24. A method of controlling the density of functional groups or functional moieties on a surface of a carbon nanostructure comprising the steps of:
providing a carbon nanostructure having a first protective layer on a surface of the structure;
forming a functional second layer over the first protective layer, the second layer having a controllable density of functional groups or functional moieties,
wherein the density of functional groups or functional moieties is controlled by applying bipolar molecules having a predetermined ratio of functional groups or functional moieties.

25. The method of claim 24 wherein the step of forming the second layer further comprises mixing a first polyoxyethylene alkyl ether including a first functional group or moiety with a second polyoxyethylene alkyl ether including a second functional group or moiety.

26. The method of claim 25 wherein the first polyoxyethylene alkyl ether is derivatized with a terminal —NH₂ or —NH— group or a polyoxyethylene alkyl ether anthraquinone 2-carboxylic acid conjugate.

27. The method of claim 25 wherein the second polyoxyethylene alkyl ether is a non-derivatized polyoxyethylene alkyl ether.

28. The method of claim 24 wherein the step of providing a carbon nanostructure further comprises contacting the carbon nanostructures with a composition comprising an alkyl protective moiety under conditions that permit the formation of an alkyl protective layer.

29. The method of claim 28 wherein the alkyl protective moiety is selected from the group consisting of linear alkanes, branched alkanes, alkenes, alkenes containing 10 to 50 carbon atoms, alkenes substituted with one or more halogen atoms, n-octadecane, n-dodecane, eicosane and hexatriacontane, and combinations thereof.

30. The method of claim 24 wherein the functional groups or moieties are selected the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof.

31. The method of claim 24 further comprising the step of adding further functional groups or functional moieties selected from the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof,
wherein the step of adding is dictated by the composition of the second layer.

32. A method of modulating hydrophilicity of a carbon nanostructure comprising the steps of:
providing a carbon nanostructure having a first protective layer on a surface of the structure; and
forming a hydrophilic second layer over the first protective layer using compounds having one or more —OH groups, —NH₂ groups or —NH— groups.

33. The method of claim 32 wherein the step of forming a hydrophilic second layer further comprises depositing molecules selected from the group consisting of C12EG30, (2,2,2-trimethylol)ethylamine tri(3,6,9,12-tetraoxaicosanyl)ether, and dioctadecylamine [(n-C₁₈H₃₈)₂NH].

34. The method of claim 32 wherein the step of providing a carbon nanostructure further comprises contacting the carbon nanostructures with a composition comprising an alkyl protective moiety under conditions that permit the formation of an alkyl protective layer.

35. The method of claim 34 wherein the alkyl protective moiety is selected from the group consisting of linear alkanes, branched alkanes, alkenes, alkenes containing 10 to 50 carbon atoms, alkenes substituted with one or more halogen atoms, n-octadecane, n-dodecane, eicosane and hexatriacontane, and combinations thereof.

36. The method of claim 32 further comprising the step of covalently attaching functional groups or functional moieties to the second layer.

37. The method of claim 36 wherein the functional groups or functional moieties are selected from the group consisting of redox mediator molecules, crown ethers, catalysts, boric acids, carbohydrates, oligonucleotides, DNA apatmers, RNA aptamers, peptide aptamers, proteins, enzymes, antibodies, quantum dots, nanoparticles, cells, cell organelles, or other cellular components, and combinations thereof.
